(19) United States
(12) Patent Application Publication
(10) Pub. No.: US 2017/0055888 A1
(43) Pub. Date: Mar. 2, 2017

(54) INFORMATION PROCESSING DEVICE, INFORMATION PROCESSING METHOD, AND PROGRAM

(71) Applicant: NORITSU PRECISION CO., LTD., Wakayama-shi, Wakayama (JP)

(72) Inventors: Shuichi Matsumoto, Wakayama-shi (JP); Takeshi Murai, Wakayama-shi (JP); Akinori Saeki, Wakayama-shi (JP); Yumiko Nakagawa, Wakayama-shi (JP); Masayoshi Uetsuji, Wakayama-shi (JP)

(73) Assignee: NORITSU PRECISION CO., LTD., Wakayama-shi, Wakayama (JP)

(21) Appl. No.: 15/118,714

(22) PCT Filed: Jan. 22, 2015
(86) PCT No.: PCT/JP2015/051633

§ 371 Date: Aug. 12, 2016

(30) Foreign Application Priority Data
Feb. 18, 2014 (JP) 2014-028656

(51) Int. Cl. A61B 5/11; H04N 7/18; G06T 7/00; G06K 9/00; H04N 5/232

(52) U.S. Cl. CPC A61B 5/1128; A61B 5/1115; A61B 5/1116; G06K 9/00771; G06K 9/00335; H04N 5/23293; H04N 5/23216; G06T 7/0051; G06T 7/0044; G06T 7/0081; H04N 7/183; G06T 7/0085; G06T 2207/10028; G06T 2207/30232; G06T 2207/20144; G06T 2207/30196

(57) ABSTRACT

The present invention provides an information processing device that, when behavior to be watched for is selected by a behavior selection unit, displays a candidate arrangement position of an image capturing device that depends on the selection on a screen. Thereafter, the information processing device detects the behavior selected to be watched for, by determining whether the positional relationship between a person being watched over and a bed satisfies a predetermined condition.

### TECHNICAL FIELD

The present invention relates to an information processing device, an information processing method, and a program.

### BACKGROUND ART

There is a technology that judges an in-bed event and an out-of-bed event, by respectively detecting human body movement from a floor region to a bed region and detecting human body movement from the bed region to the floor region, passing through a boundary edge of an image captured diagonally downward from an upward position inside a room (Patent Literature 1).

Also, there is a technology that sets a watching region for determining that a patient who is sleeping in bed has carried out a getting up action to a region directly above the bed that includes the patient who is in bed, and judges that the patient has carried out the getting up action, in the case where a variable indicating the size of an image region that the patient is thought to occupy in the watching region of a captured image that includes the watching region from a lateral direction of the bed is less than an initial value indicating the size of an image region that the patient is thought to occupy in the watching region of a captured image obtained from a camera in a state in which the patient is sleeping in bed (Patent Literature 2).

### CITATION LIST

### Patent Literature

Patent Literature 1: JP 2002-230533A

Patent Literature 2: JP 2011-005171A

### SUMMARY OF INVENTION

### Technical Problem

In recent years, accidents involving people who are being watched over such as inpatients, facility residents and care-receivers rolling or falling from bed, and accidents caused by the wandering of dementia patients have tended to increase year by year. As a method of preventing such accidents, watching systems, such as illustrated in Patent Literatures 1 and 2, for example, that detect the behavior of a person who is being watched over, such as sitting up, edge sitting and being out of bed, by capturing the person being watched over with an image capturing device (camera) installed in the room and analyzing the captured image have been developed.

In the case where the behavior in bed of a person being watched over is watched over by such as a watching system, the watching system detects various behavior of the person being watched over based on the relative positional relationship between the person being watched over and the bed, for example. Thus, when the arrangement of the image capturing device with respect to the bed changes due to a change in the environment in which watching over is performed (hereinafter, also referred to as the “watching environment”), the watching system may possibly be no longer able to appropriately detect the behavior of the person being watched over.

In order to avoid such a situation, setting of the watching system needs to be performed appropriately. However, such setting has conventionally been performed by an administrator of the system, and a user who had poor knowledge regarding the watching system was not easily able to perform setting of the watching system.

The present invention was, in one aspect, made in consideration of such points, and it is an object thereof to provide a technology that enables setting of a watching system to be easily performed.

### Solution to Problem

The present invention employs the following configurations in order to solve the abovementioned problem.

That is, an information processing device according to one aspect of the present invention includes a behavior selection unit configured to accept selection of behavior to be watched for with regard to a person being watched over, from a plurality of types of behavior, related to a bed, of the person being watched over, a display control unit configured to cause a display device to display a candidate arrangement position, with respect to the bed, of an image capturing device for watching for behavior, in the bed, of the person being watched over, according to the behavior selected to be watched for, an image acquisition unit configured to acquire a captured linkage captured by the image capturing device, and a behavior detection unit configured to detect the behavior selected to be watched for, by determining whether a positional relationship between the bed and the person being watched over appearing in the captured image satisfies a predetermined condition.

According to the above configuration, the behavior in bed of the person being watched over is captured by an image capturing device. The information processing device according to the above configuration detects the behavior of the person being watched over, utilizing the captured image that is acquired by this image capturing device. Thus, when the arrangement of the image capturing device with respect to the bed changes due to the watching environment changing, the information processing device according to the above configuration may possibly be no longer able to appropriately detect the behavior of the person being watched over.

In view of this, the information processing device according to the above configuration accepts selection of behavior to be watched for regarding the person being watched over from a plurality of types of behavior of the person being watched over that are related to the bed. The information processing device according to the above configuration then displays, on a display device, candidate arrangement positions, with respect to the bed, of an image capturing device for watching for behavior in bed of the person being watched over, according to the behavior selected to be watched for.

The user thereby becomes able to arrange the image capturing device in a position from which the behavior of the person being watched over can be appropriately detected, by arranging the image capturing device in accordance with the candidate arrangement positions of the image capturing device that are displayed on the display device. In other words, even a user who has poor knowledge of the watching system becomes able to appropriately set the watching system, at least with regard to arranging the image capturing device, simply by arranging the image capturing device in accordance with the candidate arrangement positions of the image capturing device that are displayed on the display device. Therefore, according to the above configuration, it becomes possible to easily perform setting of the watching system. Note that the person being watched over is a person whose behavior in bed is watched over using the present invention, such as an inpatient, a facility resident or a care-receiver, for example.

Also, as another mode of the information processing device according to the above aspect, the display control unit may cause the display device to further display a preset position where installation of the image capturing device is not recommended, in addition to the candidate arrangement position of the image capturing device with respect to the bed. According to this configuration, possible arrangement positions of the image capturing device that are shown as candidate arrangement positions of the image capturing device become more clearly evident, as a result of positions where installation of the image capturing device is not recommended being shown. The possibility of the user erroneously arranging the image capturing device can thereby be reduced.

Also, as another mode of the information processing device according to the above aspect, the display control unit, after accepting that arrangement of the image capturing device has been completed, may cause the display device to display the captured image acquired by the image capturing device, together with instruction content for aligning orientation of the image capturing device with the bed. With this configuration, the user is instructed in different steps as to arrangement of the camera and adjustment of the orientation of the camera. Thus, it becomes possible for the user to appropriately arrange the camera and adjust the orientation of the camera in order. Accordingly, this configuration enables even a user who has poor knowledge of the watching system to easily perform setting of the watching system.

Also, as another mode of the information processing device according to the above aspect, the image acquisition unit may acquire a captured image including depth information indicating a depth for each pixel within the captured image. Also, the behavior defection unit may detect the behavior selected to be watched for, by determining whether a positional relationship within real space between the person being watched over and a region of the bed satisfies a predetermined condition, based on the depth for each pixel within the captured image that is indicated by the depth information, as the determination of whether the positional relationship between the bed and the person being watched over appearing in the captured image satisfies a predetermined condition.

According to this configuration, depth information indicating the depth for each pixel is included in the captured image that is acquired by the image capturing device. The depth for each pixel indicates the depth of the target appearing in that pixel. Thus, by utilizing this depth information, the positional relationship in real space of the person being watched over with respect to the bed can be inferred, and the behavior of the person being watched over can be detected.

In view of this, the information processing device according to the above configuration determines whether the positional relationship within real space between the person being watched over and the bed region satisfies a predetermined condition, based on the depth for each pixel within the captured image. The information processing device according to the above configuration then infers the positional relationship within real space between the person being watched over and the bed, based on the result of this determination, and detects behavior of the person being watched over that is related to the bed.

It thereby becomes possible to detect behavior of the person being watched over with consideration for the state within real space. With the above configuration that infers the state in real space of the person being watched over utilizing depth information, however, the image capturing device has to be arranged with consideration for the depth information that is acquired, and thus it is difficult to arrange the image capturing device in an appropriate position. Thus, with the above configuration that infers the behavior of the person being watched over utilizing depth information, the present technology that facilitates setting of the watching system by displaying candidate arrangement positions of the image capturing device to prompt the user to arrange the image capturing device in an appropriate position is important.

Also, as another mode of the information processing device according to the above aspect, the information processing device may further include a setting unit configured to, after accepting that arrangement of the image capturing device has been completed, accept designation of a height of a reference plane of the bed, and set the designated height as the height of the reference plane of the bed. Also, the display control unit, when the setting unit is accepting designation of the height of the reference plane of the bed, may cause the display device to display the captured image that is acquired, so as to clearly indicate, on the captured image, a region capturing a target located at the height designated as the height of the reference plane of the bed, based on the depth for each pixel within the captured image that is indicated by the depth information, and the behavior detection unit may detect the behavior selected to be watched for, by determining whether a positional relationship between the reference plane of the bed and the person being watched over in a height direction of the bed within real space satisfies a predetermined condition.

With the above configuration, setting of the height of the reference plane of the bed is performed, as setting relating to the position of the bed for specifying the position of the bed within real space. While this setting of the height of the reference plane of the bed is performed, the information processing device according to the above configuration clearly indicates, on the captured image that is displayed on the display device, a region capturing the target, that, is located at the height that has been designated by the user. Accordingly, the user of this information processing device is able to set the height of the reference plane of the bed, while checking, on the captured image that is displayed on the display device, the height of the region designated as the reference plane of the bed.

Therefore, according to the above configuration, it is possible, even for a user who has poor knowledge of the watching system, to easily perform setting relating to the position of the bed that serves as a reference for detecting the behavior of the person being watched over, and to easily perform setting of the watching system.

Also, as another mode of the information processing device according to the above aspect, the information processing device may further include a foreground extraction unit configured to extract a foreground region of the captured image from a difference between the captured image and a background image set as a background of the captured image. Also, the behavior detection unit may detect the behavior selected to be watched for, by determining whether the positional relationship between the reference plane of the bed and the person being watched over in the height direction of the bed within real space satisfies a predetermined condition, utilizing, as a position of the person being watched over, a position within real space of a target appearing in the foreground region that is specified based on the depth for each pixel within the foreground region.

According to this configuration, a foreground region of the captured image is specified, by extracting the difference between a background image and the captured image. This foreground region is a region in which change has occurred from the background image. Thus, the foreground region includes, as an image related to the person being watched over, a region in which change has occurred due to movement of the person being watched over, or in other words, a region in which there exists a part of the body of the person being watched over that has moved (hereinafter, also referred to as the “moving part”). Therefore, by referring to the depth for each pixel within the foreground region that is indicated by the depth information, it is possible to specify the position of the moving part of the person being watched over within real space,

In view of this, the information processing device according to the above configuration determines whether the positional relationship between the reference plane of the bed and the person being watched over satisfies a predetermined condition, utilizing the position within real space of a target appearing in the foreground region that is specified based on the depth for each pixel within the foreground region as the position of the person being watched over. That is, the predetermined condition for detecting the behavior of the person being watched over is set assuming that the foreground region is related to the behavior of the person being watched over. The information processing device according to the above configuration detects the behavior of the person being watched over, based on the height at which the moving part of the person being watched over exists with respect to the reference plan of the bed within real space.

Here, the foreground region can be extracted with the difference between the background image and the captured image, and can thus be specified without using advanced image processing. Thus, according to the above configuration, it becomes possible to detect the behavior of the person being watched over with a simple method.

Also, as another mode of the information processing device according to the above aspect, the behavior selection unit may accept selection of behavior to be watched for with regard to the person being watched over, from a plurality of types of behavior, related to the bed, of the person being watched over that include predetermined behavior of the person being watched over that is carried out in proximity to or on an outer side of an edge portion of the bed. Also, the setting unit may accept designation, of a height of a bed upper surface as the height of the reference plane of the bed and set the designated height as the height of the bed upper surface, and may, in a case where the predetermined behavior is included in the behavior selected to be watched for, further accept, after setting the height of the bed upper surface, designation, within the captured image, of an orientation of the bed and a position of a reference point that is set within the bed upper surface in order to specify a range of the bed upper surface, and set a range within real space of the bed upper surface based on the designated orientation of the bed and position of the reference point. Furthermore, the behavior detection unit may detect the predetermined behavior selected to be watched for, by determining whether a positional relationship within real space between the set upper surface of the bed and the person being watched over satisfies a predetermined condition.

According to this configuration, since the range of the bed upper surface can be designated simply by designating the position of a reference point and the orientation of the bed, the range of the bed upper surface can be set with simple setting. Also, according to the above configuration, since the range of the bed upper surface is set, the detection accuracy of predetermined behavior that is carried out in proximity to or on the outer side of an edge portion of the bed can be enhanced. Note that predetermined behavior of the person being watched that is carried out in proximity to or on the outer side of an edge portion of the bed includes edge sitting, being over the rails, and being out of bed, for example. Here, edge sitting refers to a state in which the person being watched over is sitting on the edge of the bed. Also, being over the rails refers to a state in which the person being watched over is leaning out over rails of the bed.

Also, as another mode of the information processing device according to the above aspect, the behavior selection unit may accept selection of behavior to be watched for with regard to the person being watched over, from a plurality of types of behavior, related to the bed, of the person being watched over that include predetermined behavior of the person being watched over that, is carried out in proximity to or on an outer side of an edge portion of the bed. Also, the setting unit may accept designation of a height of a bed upper surface as the height of the reference plane of the bed and sets the designated height as the height of the bed upper surface, and may, in a case where the predetermined behavior is included in the behavior selected to be watched for, further accept, after set ting the height of the bed upper surface, designation, within the captured image, of positions of two corners out of four corners defining a range of the bed upper surface, and set a range within real space of the bed upper surface based on the designated positions of the two corners. Furthermore, the behavior detection unit may detect the predetermined behavior selected to be watched for, by determining whether a positional relationship within real space between the set upper surface of the bed and the person being watched over satisfies a predetermined condition. According to this configuration, since the range of the bed upper surface can be designated simply by designating the position of two corners of the bed upper surface, the range of the bed upper surface can set with simple setting. Also, according to this configuration, since the range on the upper surface of the bed is set, the detection accuracy of predetermined behavior that is carried out in proximity to or on the outer side of an edge portion of the bed can be enhanced.

Also, as another mode of the information processing device according to the above aspect, the setting unit may determine, with respect to the set range of the bed upper surface, whether a detection region specified based on the predetermined condition set in order to detect the predetermined behavior selected to be watched for appears within the captured image, and may, in a case where it is determined that the detection region of the predetermined behavior selected to be watched for does not appear within the captured image, output a warning message indicating that there is a possibility that detection of the predetermined behavior selected to be watched for cannot be performed normally. According to this configuration, erroneous setting of the watching system can be prevented, with respect to behavior selected to be watched for.

Also, as another mode of the information processing device according to the above aspect, the information processing device may further include a foreground extraction unit configured to extract a foreground region of the captured image from a difference between the captured image and a background image set as a background of the captured image.

Also, the behavior detection unit may detect the predetermined behavior selected to be watched for, by determining whether a positional relationship within real space between the bed upper surface and the person being watched over satisfies a predetermined condition, utilizing, as a position of the person being watched over, a position within real space of a target appearing in the foreground region that is specified based on the depth for each pixel within the foreground region. According to this configuration, it becomes possible to detect the behavior of the person being watched over with a simple method.

Also, as another mode of the information processing device according to the above aspect, the information processing device may further include a non-completion notification unit configured to, in a case where setting by the setting unit is not completed within a predetermined period of time, perform notification for informing that setting by the setting unit has not been completed. According to this configuration, it becomes possible to prevent the watching system from being left with setting relating to the position of the bed partially completed.

Note that as another mode of the information processing device according to each of the above modes, the present invention may be an information processing system, an information processing method, or a program that realizes each of the above configurations, or may be a storage medium having such a program recorded thereon and readable by a computer or other device, machine or the like. Here, a storage medium that is readable by a computer or the like is a medium that stores information such as programs by an electrical, magnetic, optical, mechanical or chemical action. Also, the information processing system may be realized by one or a plurality of information processing devices.

For example, an information processing method according to one aspect of the present invention is an information processing method in which a computer executes a step of accepting selection of behavior to be watched for with regard to a person being watched over, from a plurality of types of behavior, related to a bed, of the person being watched over, a step of causing a display device to display a candidate arrangement position, with respect to the bed, of an image capturing device for watching for behavior, in the bed, of the person being watched over, according to the behavior selected to be watched for, a step of acquiring a captured image captured by the image capturing device, and a step of defecting the behavior selected to be watched for, by determining whether a positional relationship between the bed and the person being watched over appearing in the captured image satisfies a predetermined condition,

Also, for example, a program according to one aspect of the present invention is a program for causing a computer to execute a step of accepting selection of behavior to be watched for with regard to a person being watched over, from a plurality of types of behavior, related to a bed, of the person being watched over, a step of causing a display device to display a candidate arrangement position, with respect to the bed, of an image capturing device for watching for behavior, in the bed, of the person being watched over, according to the behavior selected to be watched for, a step of acquiring a captured image captured by the image capturing device, and a step of defecting the behavior selected to be watched for, by determining whether a positional relationship between the bed and the person being watched over appearing in the captured image satisfies a predetermined condition.

### Advantageous Effects of Invention

According to the present invention, it becomes possible to easily perform setting of a watching system.

### BRIEF DESCRIPTION OF DRAWINGS

FIG. 1 shows an example of a situation in which the present invention is applied.

FIG. 2 shows an example of a captured image in which a gray value of each pixel is determined according to the depth for that pixel.

FIG. 3 illustrates a hardware configuration of an information processing device according to an embodiment.

FIG. 4 illustrates depth according to the embodiment.

FIG. 5 illustrates a functional configuration according to the embodiment.

FIG. 6 illustrates a processing procedure by the information processing device when performing setting relating to the position of a bed in the present embodiment.

FIG. 7 illustrates a screen for accepting selection of behavior to be detected.

FIG. 8 illustrates candidate camera arrangement positions that are displayed on a display device, in the case where out-of-bed is selected as behavior to be detected.

FIG. 9 illustrates a screen for accepting designation of the height of a bed upper surface.

FIG. 10 illustrates the coordinate relationship within a captured image.

FIG. 11 illustrates the positional relationship within real space between the camera and arbitrary points (pixels) of a captured image.

FIG. 12 schematically illustrates regions that are displayed in different display modes within a captured image.

FIG. 13 illustrates a screen for accepting designation of the range on the bed upper surface.

FIG. 14 illustrates the positional relationship between a designated point on a captured image and a reference point of the bed upper surface.

FIG. 15 illustrates the positional relationship between the camera and the reference point.

FIG. 16 illustrates the positional relationship between the camera and the reference point.

FIG. 17 illustrates the relationship between a camera coordinate system and a bed coordinate system.

FIG. 18 illustrates a processing procedure by the information processing device when detecting the behavior of a person being watched over in the embodiment.

FIG. 19 illustrates a captured image that is acquired by the information processing device according to the embodiment.

FIG. 20 illustrates the three-dimensional distribution of a subject in an image capturing range that is specified based on depth information that is included in a captured image.

FIG. 21 illustrates the three-dimensional distribution of a foreground region that is extracted from a captured image.

FIG. 22 schematically illustrates a detection region for detecting sitting up in the embodiment.

FIG. 23 schematically illustrates a detection region for detecting being out of bed in the embodiment.

FIG. 24 schematically illustrates a detection region for detecting edge sitting in the embodiment.

FIG. 25 illustrates the relationship between dispersion and the degree of spread of a region.

FIG. 26 shows another example of a screen for accepting designation of the range of the bed upper surface.

### DESCRIPTION OF EMBODIMENTS

Hereinafter, an embodiment (hereinafter, also described as “the present embodiment”) according to one aspect of the present invention will be described based on the drawings. The present embodiment described below is, however, to be considered in ail respects as illustrative of the present invention. It is to be understood that various improvements and modifications can be made without departing from the scope of the present invention. In other words, in implementing the present invention, specific configurations that depend on the embodiment may be employed as appropriate.

Note that data appearing in the present embodiment will be described using natural language, and will, more specifically, be designated with computer-recognizable quasi-language, commands, parameters, machine language, and the like.

### 1. Exemplary Application Situation

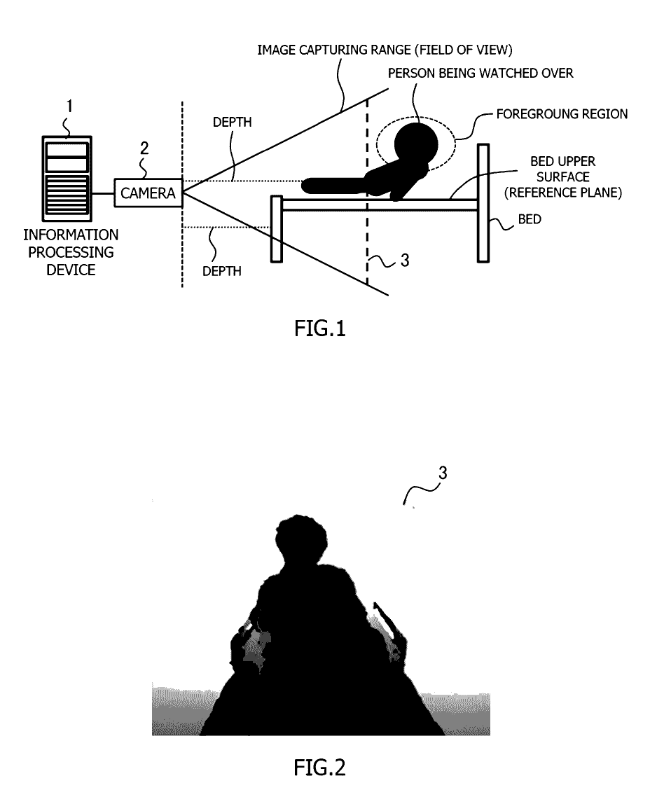

First, a situation to which the present invention is applied will be described using FIG. 1. FIG. 1 schematically shows an example of a situation to which the present invention is applied. In the present embodiment, a situation in which the behavior of an inpatient or a facility resident is watched over in a medical facility or a nursing facility is assumed as a person being watched over. The person who watches over the person being watched over (hereinafter, also referred to as the “user”) watches over the behavior in bed of a person being watched over, utilizing a watching system that includes an information processing device 1 and a camera 2.

The watching system according to the present embodiment acquires a captured image 3 in which the person being watched over and the bed appear, by capturing the behavior of the person being watched over using the camera 2. The watching system then detects the behavior of the person being watched over, by using the information processing device 1 to analyze the captured image 3 that is acquired with the camera 2.

The camera 2 corresponds to an image capturing device of the present invention, and is installed in order to watch over the behavior in bed of the person being watched over. The camera 2 according to a present embodiment includes the depth sensor that measures the depth of a subject, and is able to acquire the depth corresponding to each pixel within a captured image. Thus, the captured image 3 that is acquired by this camera 2 includes depth information indicating the depth obtained for every pixel, as illustrated in FIG. 1.

This captured image 3 including depth information may be data indicating the depth of a subject within the image capturing range, or may be data in which the depth of a subject within the image capturing range is distributed two-dimensionally (e.g., depth map), for example. Also, the captured image 3 may include an RGB image together with depth information. Furthermore, the captured image 3 may be a moving image or may be a static image.

FIG. 2 shows an example of such a captured image 3. The captured image 3 illustrated in FIG. 2 is an image in which the gray value of each pixel is determined according to the depth for that pixel. Blacker pixels indicate decreased distance to the camera 2. On the other hand, whiter pixels indicate increased distance to the camera 2. This depth information enables the position within real space (three-dimensional space) of the subject within the image capturing range to be specified.

More specifically, the depth of a subject is acquired with respect to the surface of that subject. The position within real space of the surface of the subject captured on the camera 2 can then be specified, by using the depth information that is included in the captured image 3. In the present embodiment, the captured image 3 captured by the camera 2 is transmitted to the information processing device 1. The information processing device I then infers the behavior of the person being watched over, based on the acquired captured image

The information processing device 1 according to the present embodiment specifies a foreground region within the captured image 3, by extracting the difference between the captured image 3 and a background image that is set as the background of the captured image 3, in order to infer the behavior of the person being watched over based on the captured image 3 that is acquired. The foreground region that is specified is a region in which change has occurred from the background image, and thus includes the region in which the moving part of the person being watched over exists. In view of this, the information processing device 1 detects the behavior of the person being watched over, utilizing the foreground region as an image related to the person being watched over.

For example, in the case where the person being watched over sits up in bed, the region in which the part relating to the sitting up (upper body in FIG. 1) appears is extracted as the foreground region, as illustrated in FIG. 1. It is possible to specify the position of the moving part of the person being watched over within real space, by referring to the depth for each pixel within the foreground region that is thus extracted.

It is possible to infer the behavior in bed of the person being watched over based on the positional relationship between the moving part that is thus specified and the bed. For example, in the case where the moving part of the person being watched over is detected upward of the upper surface of the bed, as illustrated in FIG. 1, it can be inferred that the person being watched over has carried out the movement of sitting up in bed. Also, in the case where the moving part of the person being watched over is detected in proximity to the side of the bed, for example, it can be inferred that the person being watched over is moving to an edge sitting state.

In view of this, the information processing device 1 according to the present embodiment detects the behavior of the person being watched over, based on the positional relationship within real space between the target appearing in the foreground region and the bed. In other words, the information processing device 1 utilizes the position within real space of a target appearing in the foreground region that is specified based on the depth for each pixel within the foreground region as the position of the person being watched over. The information processing device 1 then detects the behavior of the person being watched over, based on where, within real space, the moving part of the person being watched over is positioned with respect to the bed. Thus, the information processing device 1 according to the present embodiment may no longer be able to appropriately detect the behavior of the person being watched over when the arrangement of the camera 2 with respect to the bed changes due to the watching environment changing.

In order to address this, the information processing device 1 according to the present embodiment accepts selection of behavior to be watched for regarding the person being watched over from a plurality of types of behavior of the person being watched over that are related to the bed. The information processing device 1 then displays, on a display device, candidate arrangement positions of the camera 2 with respect to the bed, according to the behavior selected to be watched for.

The user thereby becomes able to arrange the camera 2 in a position from which the behavior of the person being watched over can be appropriately detected, by arranging the camera 2 in accordance with candidate arrangement positions of the camera 2 that are displayed on the display device. In other words, even a user who has poor knowledge of the watching system becomes able to appropriately set the watching system, simply by arranging the camera 2 in accordance with candidate arrangement positions of the camera 2 that are displayed on the display device. Thus, according to the present embodiment, it becomes possible to easily perform setting of the watching system.

Note that, in FIG. 1, the camera 2 is arranged forward of the bed in the longitudinal direction. That is, FIG. 1 illustrates a situation in which the camera 2 is viewed from the side, and the up-down direction in FIG. I corresponds to the height direction of the bed. Also, the left-right direction in FIG. 1 corresponds to the longitudinal direction of the bed, and the direction perpendicular to the page in FIG. 1 corresponds to the width direction of the bed. The position in which the camera 2 can be arranged is, however, not limited to such a position, and may be selected, as appropriate, according to the embodiment. The user becomes able to arrange the camera 2 in an appropriate position to detect the behavior selected to be watched for, among possible arrangement positions of the camera 2 thus selected as appropriate, by arranging the camera 2 in accordance with display content on the display device.

Also, in the information processing device 1 according to the present embodiment, setting of the reference plane of the bed, for specifying the position of the bed within real space. is performed so as to be able to grasp the positional relationship between the moving part and the bed. In the present embodiment, the upper surface of the bed is employed as this reference plane of the bed. The bed upper surface is the surface of the upper side of the bed in the vertical direction, and is, for example, the upper surface of the bed mattress. The reference plane of the bed may be such a bed upper surface, or may be another surface. The reference plane of the bed may be decided, as appropriate, according to the embodiment. Also, the reference plane of the bed may be not only a physical surface existing on the bed but a virtual surface.

### 2. Exemplary Configuration

### Exemplary Hardware Configuration

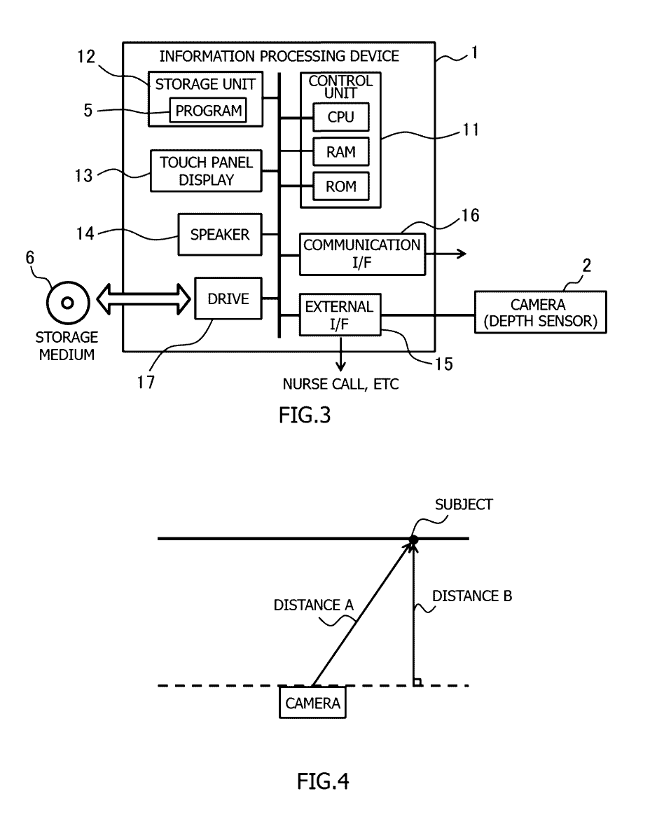

Next, the hardware configuration of the information processing device 1 will be described using FIG. 3. FIG. 3 illustrates the hardware configuration of the information processing device 1 according to the present embodiment. The information processing device 1 is a computer in which a control unit 11 including a CPU, a RAM (Random Access Memory), a ROM (Read Only Memory) and the like, a storage unit 12 storing information such as a program 5 that is executed by the control unit 11, a touch panel display 13 for performing image display and input, a speaker 14 for outputting audio, an external interface 15 for connecting to an external device, a communication interface 16 for performing communication via a network, and a drive 17 for reading programs stored in a storage medium 6 are electrically connected, as illustrated in FIG. 3. In FIG. 3, the communication interface and the external interface are respectively described as a “communication I/F” and an “external I/F”.

Note that, in relationship to the specific hardware configuration of the information processing device 1, constituent elements can be omitted, replaced or added, as appropriate, according to the embodiment. For example, the control unit 11 may include a plurality of processors. Also, for example, the touch panel display 13 may be replaced by an input device and a display device that are respectively separately connected independently.

The information processing device 1 may be provided with a plurality of external interfaces 15, and may be connected to a plurality of external devices. In the present embodiment, the information processing device 1 is connected to the camera 2 via the external interface 15. The camera 2 according to the present embodiment includes a depth sensor, as described above. The type and measurement method of this depth sensor may be selected as appropriate according to the embodiment.

The place (e.g., ward of a medical facility) where watching over of the person being watched over is performed, however, is a place where the bed of the person being watched over is located, or in other words, the place where the person being watched over sleeps. Thus, the place where watching over of the person being watched over is performed is often a dark place. In view of this, in order to acquire the depth without being affected by the brightness of the place where image capture is performed,, a depth sensor that measures depth based on infrared irradiation is preferably used. Note that Kinect by Microsoft Corporation, Xtion by Asus and Carmine by PrimeSense can be given as comparatively cost-effective image capturing devices that include an infrared depth sensor.

Also, the camera 2 may be a stereo camera, so as to enable the depth of the subject within the image capturing range to be specified. The stereo camera captures the subject within the image capturing range from a plurality of different directions, and is thus able to record the depth of the subject. The camera 2 may, if the depth of the subject within the image capturing range can be specified, be replaced by a stand-alone depth sensor, and is not particularly limited.

Here, the depth measured by a depth sensor according to the present embodiment will be described in detail using FIG. 4. FIG. 4 shows an example of the distances that can be treated as a depth according to the present embodiment. This depth represents the depth of a subject. As illustrated in FIG. 4, the depth of the subject may be represented in a distance A of a straight line between the camera and the object, or may be represented in a distance B of a perpendicular down from the horizontal axis of the camera with respect to the subject, for example. That is, the depth according to the present embodiment may be the distance A or may be the distance B. In the present embodiment, the distance B will be treated as the depth. However, the distance A and the distance B are exchangeable with each other using Pythagorean theorem or the like, for example. Thus, the following description using the distance B can be directly applied to the distance A.

Also, the information processing device 1 is connected to the nurse call via the external interface 15, as illustrated in FIG. 3. In this way, the information processing device 1, by being connected to equipment installed in the facility such as a nurse call via the external interface 15, performs notification for informing that there is an indication that the person being watched over is in impending danger, in cooperation with that equipment.

Note that the program 5 is a program for causing the information processing device 1 to execute processing that is included in operations discussed later, and corresponds to a “program” of the present invention. This program 5 may be recorded in the storage medium 6. The storage medium 6 is a medium that stores programs and other information by an electrical, magnetic, optical, mechanical or chemical action, such that the programs and other information are readable by a computer or other device, machine or the like. The storage medium 6 corresponds to a “storage medium” of the present invention. Note that FIG. 3 illustrates a disk-type storage medium such as a CD (Compact Disk) or a DVD (Digital Versatile Disk) as an example of the storage medium 6. However, the storage medium 6 is not limited to a disk-type storage medium, and may be a non-disk-type storage medium. Semiconductor memory such as flash memory can be given, for example, as a non-disk-type storage medium.

Also, for example, apart from a device exclusively designed for a service that is provided, a general-purpose device such as a PC {Personal Computer) or a tablet terminal may be used as the information processing device 1. Also, the information processing device 1 may be implemented using one or a plurality of computers,

### Exemplary Functional Configuration

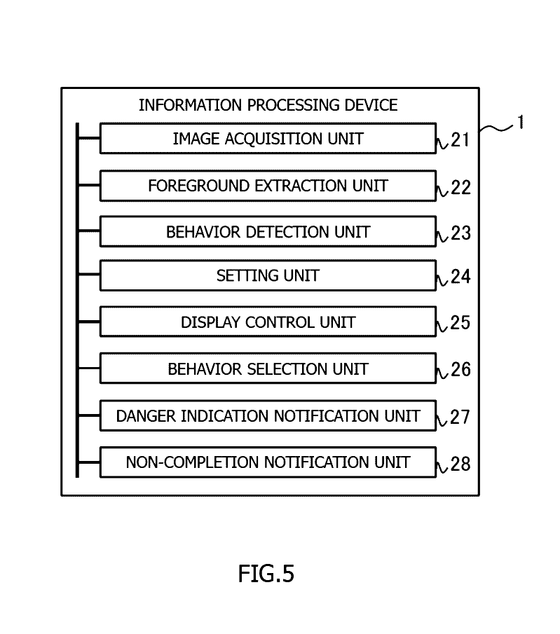

Next, the functional configuration of the information processing device 1 will be described using FIG. 5. FIG. 5 illustrates the functional configuration of the information processing device 1 according to the present embodiment. The control unit 11 with which the information processing device 1 according to the present embodiment is provided expands the program 5 stored in the storage unit 12 in the RAM. The control unit 11 then controls the constituent elements by using the CPU to interpret and execute the program 5 expanded in the RAM. The information processing device 1 according to the present embodiment thereby functions as a computer that is provided with an image acquisition unit 21, a foreground extraction unit 22, a behavior detection unit 23, a setting unit 24, a display control unit 25, a behavior selection unit 26, a danger indication notification unit 27, and a non-completion notification unit 28.

The image acquisition unit 21 acquires a captured image 3 captured by the camera 2 that is installed in order to watch over the behavior in bed of the person being watched over, and including depth information indicating the depth for each pixel. The foreground extraction unit 22 extracts a foreground region of the captured image 3 from the difference between a background image set as the background of the captured image 3 and that captured image 3. The behavior detection unit 23 determines whether the positional relationship within real space between the target appearing in the foreground region and the bed satisfies a predetermined condition,, based on the depth for each pixel within the foreground region that is indicated by the depth information. The behavior detection unit 23 then detects behavior of the person being watched over that is related to the bed, based on the result of the determination.

Also, the setting unit 24 accepts input from a user and performs setting relating to the reference plane of the bed that serves as a reference for detecting the behavior of the person being watched over. Specifically, the setting unit 24 accepts designation of the height of the reference plane of the bed, and sets the designated height as the height of the reference plane of the bed. The display control unit 25 controls image display by the touch panel display 13. The touch panel display 13 corresponds to a display device of the present invention.

The display control unit. 25 controls screen display of the touch panel display 13. The display control unit 25 displays candidate arrangement positions of the camera 2 with respect to the bed on the touch panel display 13, according to the behavior selected to be watched for by the behavior selection unit 26 which will be discussed later, for example. Also, the display control unit 25, when the setting unit 24 accepts designation of the height of the reference plane of the bed, for example, display the acquired captured image 3 on the touch panel display 13, so as to clearly indicate, on the captured image 3, a region capturing the target that is located at the height that has been designated by the user, based on the depth for each pixel within the captured image 3 that is indicated by the depth information.

The behavior selection unit 26 accepts selection of behavior to be watched for with regard to the person being watched over, that is, behavior to be detected by the above behavior detection unit 23, from a plurality of types of behavior of the person being watched over that are related to the bed including predetermined behavior of the person being watched over that is performed in proximity to or on the outer side of an edge portion of the bed. In the present embodiment, sitting up in bed, edge sitting on the bed, leaning out over the rails of the bed (being over the rails) and being out of bed are illustrated as the plurality of types of behavior that are related to the bed. Of these types of behavior, edge sitting on the bed, leaning out over the rails of the bed (being over the rails) and being out of bed correspond to “predetermined behavior” of the present invention.

Note that the plurality of types of behavior of the person being watched over that are related to the bed may include predetermined behavior of the person being watched over that is carried out in proximity to or on the outer side of an edge portion of the bed. In the present embodiment, edge sitting on the bed, being over the rails of the bed (being over the rails) and being out of bed correspond to “predetermined behavior” of the present invention.

Furthermore, the danger indication notification unit 27, in the case where the behavior detected with regard to the person being watched over is behavior showing an indication that the person being watched over is in impending danger, performs notification for informing this indication. The non-completion notification unit 28, in the case where setting relating to the reference plane of the bed by the setting unit 24 is not completed within a predetermined period of time, performs notification for informing that setting by the setting unit 24 has not been completed. Note that these notifications are performed for the person watching over the person being watched over, for example. The person watching over is, for example, a nurse, a facility staff member, or the like. In the present embodiment, these notifications may be performed through a nurse call, or may be performed using the speaker 14.

Note that each function will be discussed in detail with an exemplary operation which will be discussed later. Here, in the present embodiment, an example will be described in which these functions are all realized by a general-purpose CPU. However, some or all of these functions may be realized by one or a plurality of dedicated processors. Also, in relationship to the functional configuration of the information processing device 1, functions may be omitted, replaced or added, as appropriate, according to the embodiment. For example, the setting unit 24 r the danger indication notification unit 27 and the non-completion notification unit 28 may be omitted.

### 3. Exemplary Operation

### Setting of Watching System

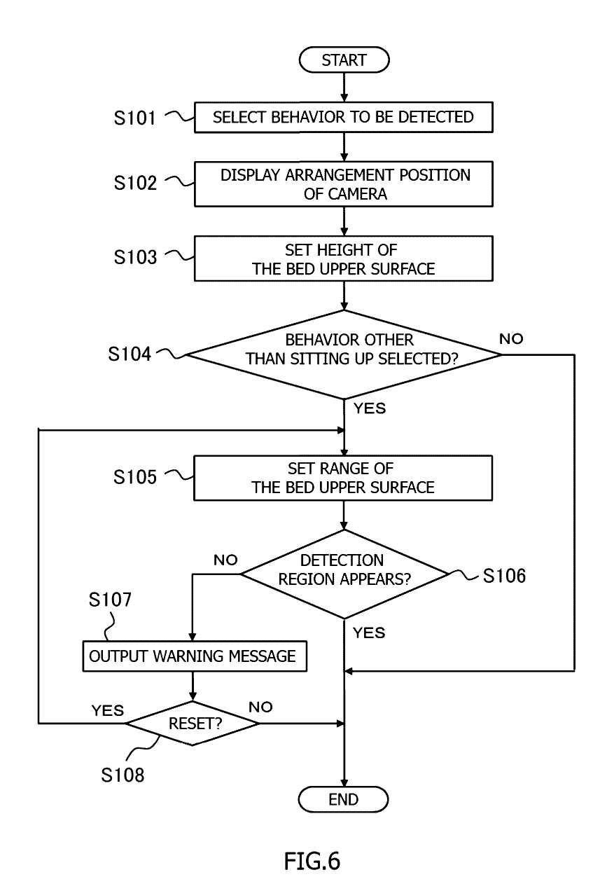

First, processing relating to setting of the watching system will be described using FIG. 6. FIG. 6 illustrates a processing procedure by the information processing device 1 when performing setting relating to the position of the bed. This processing for setting relating to the position of the bed may be performed at any timing, and is, for example, executed when the program 5 is launched, before starting watching over of the person being watched over. Note that the processing procedure described below is merely an example, and the respective processing may be modified to the full extent possible. Also, with regard to the processing procedure described below, steps can be omitted, replaced or added, as appropriate, according to the embodiment.

### Steps S101 and S102

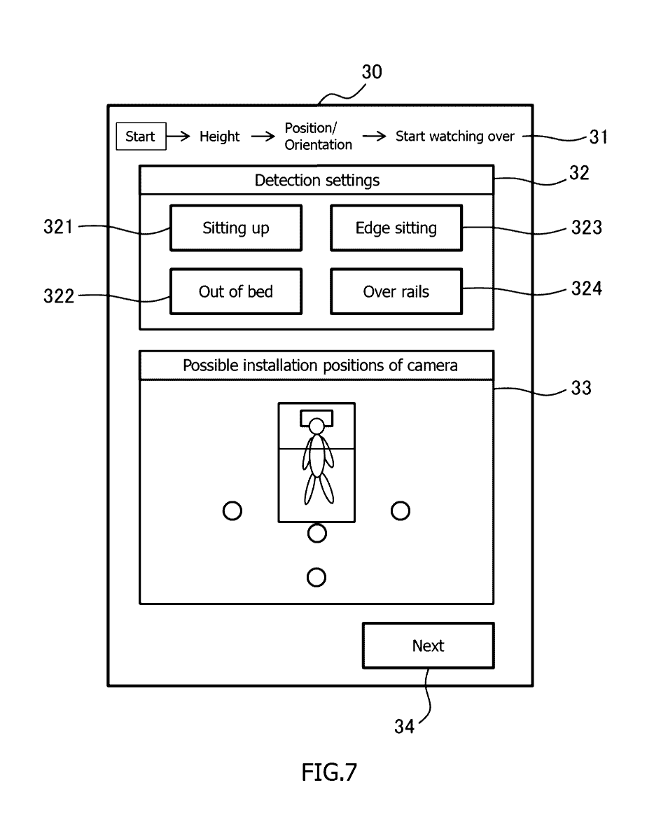

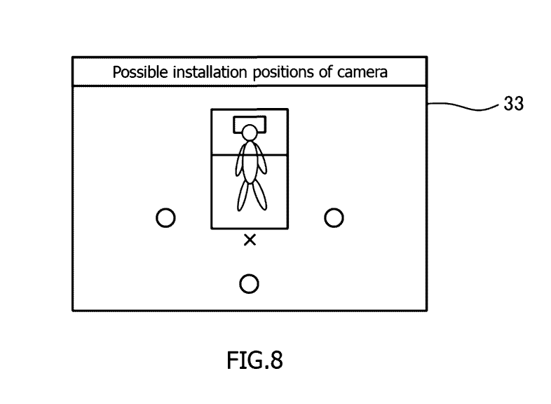

In step S101, the control unit 11 functions as the behavior selection unit 26, and accepts selection of behavior to be detected from a plurality of types of behavior that the person being watched over carries out in bed. Then in step S102, the control unit 11 functions as the display control unit 25, and causes the touch panel display 13 to display candidate arrangement positions of the camera 2 with respect to the bed, according to the one or more of types of behavior selected to be detected. The respective processing will be described using FIGS. 7 and 8.

FIG. 7 illustrates a screen 30 that is displayed on the touch panel display 13, when accepting selection of behavior to be detected. The control unit 11 displays the screen 30 on the touch panel display 13, in order to accept selection of behavior to be detected in step S101. The screen 30 includes a region 31 showing the processing stages involved in setting according to this processing, a region 32 for accepting selection of behavior to be detected, and a region 33 showing candidate arrangement positions of the camera 2.

On the screen 30 according to the present embodiment, four types of behavior are illustrated as candidate types of behavior to be detected. Specifically, sitting up in bed, being out of bed, edge sitting on the bed, and leaning out over the rails of the bed (being over the rails) are illustrated as candidate types of behavior to be detected. Hereinafter, sitting up in bed will be referred to simply as “sitting up”, being out of bed will be referred to simply as “out of bed”, edge sitting on the bed will be referred to simply as “edge sitting”, and leaning out over the rails of the bed will be referred to as “over the rails”. The four buttons 321 to 324 corresponding to the respective types of behavior are provided in the region 32 . The user selects one or more types of behavior to be detected, by operating the buttons 321 to 324.

When behavior to be detected is selected by any of the buttons 321 to 324 being operated, the control unit 11 functions as the display control unit 25, and updates the content that is displayed in the region 33, so as to show candidate arrangement positions of the camera 2 that depend on the one or more types of behavior that are selected. The candidate arrangement positions of the camera 2 are specified in advance, based on whether the information processing device 1 can detect the target behavior using the captured image 3 that is captured by the camera 2 arranged in those positions. The reasons for showing the candidate arrangement position of such a camera 2 are as follows.

The information processing device 1 according to the present embodiment infers the positional relationship between the person being watched over and the bed, and detects the behavior of the person being watched over, by analyzing the captured image 3 that is acquired by the camera 2. Thus, in the case where the region that is related to detection of the target behavior does not appear in the captured image 3, the information processing device 1 is not able to detect the target behavior. Therefore, the user of the watching system desirably has a grasp of positions that are suitable for arranging the camera 2 for every type of behavior to be detected.

However, since the user of the watching system does not necessarily grasp all of such positions, the camera 2 may possibly be erroneously arranged in a position from which the region that is related to detection of the target behavior is not captured. When the camera 2 is erroneously arranged in a position front which the region that is related to detection of the target behavior is not captured, a deficiency will occur in the watching over by the watching system, since the information processing device 1 cannot detect the target behavior.

In view of this, in the present embodiment, positions that are suitable for arranging the camera 2 are specified in advance for every type of behavior to be detected, and information relating to such candidate camera posit ions is held in the information processing device 1. The information processing device 1 then displays candidate arrangement positions of the camera 2 capable of capturing the region that is related to detection of the target behavior, according to one or more types of behavior that are selected, and instructs the user as to the arrangement position of the camera 2.

In the present embodiment, it is possible, even for a user who has poor knowledge of the watching system, to performed setting of the watching system, simply by arranging the camera 2 in accordance with candidate arrangement positions of the camera 2 displayed on the touch panel display 13. Also, by thus instructing the arrangement position of the camera 2, the camera 2 being erroneously arranged by the user is prevented, enabling the possibility of a deficiency occurring in the watching over of the person being watched over to be reduced. That is, with the watching system according to the present embodiment, it is possible, even for a user who has poor knowledge of the watching system, to easily arrange the camera 2 in an appropriate position.

Also, in the present embodiment, various settings which will be discussed later allow the degree of freedom with which the camera 2 is arranged to be increased, and enable the watching system to be adapted to various environments in which watching over is performed. However, the high degree of freedom with which the camera 2 can be arranged increases the possibility of the user arranging the camera 2 in the wrong position. In response to this, in the present embodiment, candidate arrangement positions of the camera 2 are displayed to prompt the user to arrange the camera 2, and thus the user can be prevented from arranging the camera 2 in the wrong position. That is, with a watching system in which the camera 2 is arranged with a high degree of freedom as in the present embodiment, the effect of preventing the user from arranging the camera 2 in the wrong position, by displaying candidate arrangement positions of the camera 2, can be particularly anticipated.

Note that, in the present embodiment, as candidate arrangement positions of the camera 2, positions from which the region that is related to detection of the target behavior can be easily captured by the camera 2, or in other words, positions where it is recommended to install the camera 2, are indicated with an O mark. In contrast, positions from which the region that is related to detection of the target behavior cannot be easily captured by the camera 2, or in other words, positions where it is not recommended to install the camera 2, are indicated with an X mark. A position where it is not recommended to set the camera 2 will he described using FIG. 8.

FIG. 8 illustrates the display content of the region 33 in the case where “out of bed” is selected as behavior to be detected. Being out of bed is the act of moving away from the bed. In other words, being out of bed is a movement that the person being watched over carries out on the outer side of the bed, particularly at a place away from the bed. Thus, when the camera 2 is arranged in the position from which it is difficult to capture the outer side of the bed, the possibility that the region that is related to detection of being out of bed will not appear in the captured image 3 increases.

Here, when the camera 2 is arranged in the vicinity of the bed, there is a high possibility that the captured image 3 that is captured by the camera 2 will be occupied in large part by an image in which the bed appears, and will hardly show any places away from the bed. Thus, on the screen illustrated by FIG. 8, the position in the vicinity of the bottom end of the bed is indicated with an X mark, as a position where arrangement of the camera 2 is not recommended when detecting being out of bed.

Thus, is the present embodiment, positions where arrangement of the camera 2 is not recommended are represented on the touch panel display 13, in addition to candidate arrangement positions of the camera 2. The user thereby becomes able to precisely grasp each candidate arrangement position of the camera 2, based on the positions where arrangement of the camera 2 is not recommended. Thus, according to the present embodiment, the possibility of the user erroneously arranging the camera 2 can be reduced.

Note that information (hereinafter, also referred to as “arrangement information”) for specifying candidate arrangement positions of the camera 2 that depend on the selected behavior to be detected and positions where arrangement of the camera 2 is not recommended are acquired as appropriate. The control unit 11 may, for example, acquire from the storage unit 12 this arrangement information from the storage unit 12, or from another information processing device via a network. In the arrangement information, candidate arrangement positions of the camera 2 and positions where arrangement of the camera 2 is not recommended are set in advance, according to the selected behavior to be detected, and the control unit 11 is able to specify these positions by referring to the arrangement information.

Also, the data format of this arrangement information may be selected, as appropriate, according to the embodiment. For example, the arrangement information may be data in table format that defines candidate arrangement positions of the camera 2 and positions where arrangement of the camera 2 is not recommended, for every type of behavior to be detected. Also, for example, the arrangement information may, as in the present embodiment, be data set as operations of the respective buttons 321 to 324 for selecting behavior to be detected. That is, as a mode of holding arrangement information, operations of the respective buttons 321 to 324 may be set, such that an O mark or an X mark is displayed in the candidate positions for arranging the camera 2 when the respective buttons 321 to 324 are operated.

Also, the method of representing each candidate arrangement position of the camera 2 and position where installation of the camera 2 is not recommended need not be limited to the method involving O marks and X marks illustrated in FIGS. 7 and 8, and may be selected, as appropriate, according to the embodiment. For example, the control unit 11 may display specific distances of possible arrangement positions of the camera 2 from the bed on the touch panel display 13, instead of the display content illustrated in FIGS. 7 and 8.

Furthermore, the number of the positions that are presented as candidate arrangement positions of the camera 2 and positions where installation of the camera 2 is not recommended may be set, as appropriate, according to the embodiment. For example, the control unit 11 may present a plurality of positions as candidate arrangement positions of the camera 2, or may present a single position.

In this way, in the present embodiment, when behavior that it is desired to detect is selected by the user in step S101, candidate arrangement positions of the camera 2 are shown in the region 33, according to the selected behavior to be detected, in step S102. The user arranges the camera 2, in accordance with the content in this region 33. That is, the user selects one of the candidate arrangement positions shown in the region 33, and arranges the camera 2 in the selected position, as appropriate.

A “next” button 34 is further provided on the screen 30, in order to accept that selection of behavior to be detected and arrangement of the camera 2 have been completed. The control unit 11 according to the present embodiment, as an example of a method of accepting that selection of behavior to be detected and arrangement of the camera 2 has been completed, accepts selection of behavior to be detected and that arrangement of the camera 2 has been completed, through provision of the “next” button 34 on the screen 30. When the user operates the “next” button 34 after selection of behavior to be detected and arrangement of the camera 2 have been completed, the control unit 11 of the information processing device 1 advances the processing to the next step S103.

### Step S103

Returning to FIG. 6, in step S103, the control unit 11 functions as the setting unit 24, and accepts designation of the height of the bed upper surface. The control unit 11 sets the designated height, as the height of the bed upper surface. Also, the control unit 11 functions as the image acquisition unit 21, and acquires the captured image 3 including depth information from the camera 2. The control unit 11 then functions as the display control unit 25, when accepting designation of the height of the bed upper surface, and displays the captured image 3 that is acquired on the touch panel display 13, so as to clearly indicate, on the captured linkage 3, the region capturing the target that is located at the designated height.

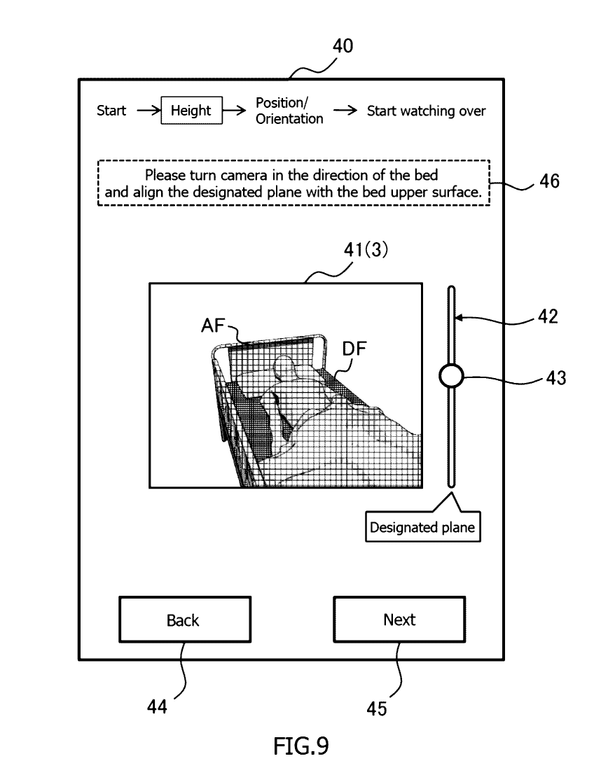

FIG. 9 illustrates a screen 40 that is displayed on the touch panel display 13 when accepting designation of the height of the bed upper surface. The control unit 11 displays the screen 40 on the touch panel display 13, in order to accept designation of the height of the bed upper surface in step S103. The screen 4 0 includes a region 41 in which the captured image 3 that, is obtained from the camera 2 is rendered, a scroll bar 42 for designating the height of the bed upper surface, and a region 46 in which instruction content for aligning the orientation of the camera 2 with the bed is rendered.

In step S102, the user has arranged the camera 2 in accordance with the content, that is displayed on the screen. In view of this, in this step S103, the control unit. 11 functions as the display control unit. 25, and renders the captured image 3 that is obtained by the camera 2 in the region 41, together with rendering the instruction content for aligning the orientation of the camera 2 with the bed in the region 46. In the present embodiment, the user is thereby instructed to adjust the orientation of the camera 2.

That is, according to the present embodiment, after being instructed as to arrangement, of the camera 2, the user can be instructed as to adjustment of the orientation of the camera. Thus, it becomes possible for the user to appropriately perform arrangement of the camera 2 and adjustment of the orientation of the camera 2 in order. Accordingly, the present embodiment enables even a user who has poor knowledge of the watching system to easily perform setting of the watching system. Note that representation of this instruction content need not be limited to the representation illustrated in FIG. 9, and may be set, as appropriate, according to the embodiment.

When the user turns the camera 2 in the direction of the bed in accordance with the instruction content rendered in the region 4 6, while checking the captured image 3 that is rendered in the region 41, such that the bed is included in the image capturing range of the camera 2, the bed will appear in the captured image 3 that is rendered in the region 41. When the bed comes to appear within the captured image 3, it becomes possible to compare the designated height and the height of the bed upper surface within the captured image 3. Thus, the user operates the knob 43 of the scroll bar 42 to designate the height of the bed upper surface, after adjusting the orientation of the camera 2.

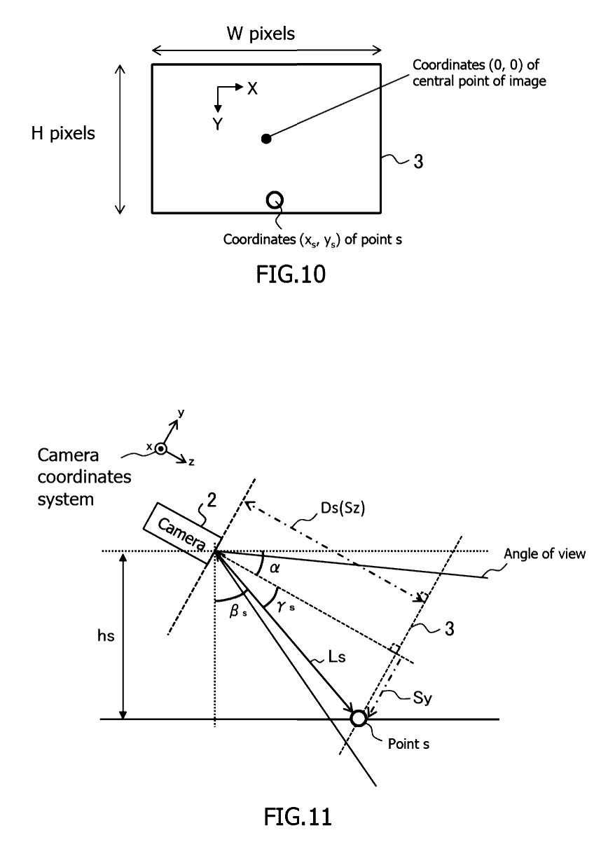

Here, the control unit 11 clearly indicates, on the captured image 3, the region capturing the target that is located at the designated height based on the position of the knob 43. The information processing device 1 according to the present embodiment thereby makes it easy for the user to grasp the height within real space that is designated based on the position of the knob 43. This processing will be described using FIGS. 10 to 12.

First, the relationship between the height of the target appearing in each pixel within the captured image 3 and the depth for that pixel will be described using FIGS. 10 and 11. FIG. 10 illustrates the coordinate relationship within the captured image 3. Also, FIG. 11 illustrates the positional relationship within, real space between an arbitrary pixel (point s) of the captured image 3 and the camera 2. Note that the left-right direction in FIG. 10 corresponds to a direction perpendicular to the page of FIG. 11. That is, the length of the captured image 3 that appears in FIG. 11 corresponds to the length (H pixel) in the vertical direction illustrated in FIG. 10. Also, the length (W pixel) in the lateral direction illustrated in FIG. 10 corresponds to the length of the captured image 3 in the direction perpendicular to the page that does not appear in FIG. 11.

Here, the coordinates of the arbitrary pixel {point s) of the captured image 3 are given as (xₛ, yₛ), as illustrated in FIG. 10, the angle of view of the camera 2 in the lateral direction is given as Vx, and the angle of view in the vertical direction is given as Vy. The number of pixels of the captured image 3 in the lateral direction is given as W, the number of pixels in the vertical direction is given as H, and the coordinates of a central point (pixel) of the captured image 3 are given as (0, 0).

Also, the pitch angle of the camera 2 is given as of, as illustrated in FIG. 11. The angle between a line segment connecting the camera 2 and the point s and a line segment indicating the vertical direction within real space is given as and the angle between the line segment connecting the camera 2 and the point s, and a line segment indicating the image capturing direction of the camera 2 is given as γₛ. Furthermore, length of the line segment connecting the camera 2 and the point s as viewed from the lateral direction is given as Lₛ, and vertical distance between the camera 2 and the point s is given as hs. Mote that, in the present embodiment, this distance hs corresponds to the height within real space of the target appearing at the point s. The method of representing the height within real space of the target appearing at the point s is, however, not limited to such an example, and may be set, as appropriate, according to the embodiment.

The control unit 11 is able to acquire information indicating an angle of view (Vₓ/V_{y}) and a pitch angle α of this camera 2 from the camera 2. The method of acquiring this information is, however, not limited to such a method, and the control unit 11 may acquire this information by accepting input from the user, or as a set value that is set in advance.

Also, the control unit 11 is able to acquire the coordinates (xₛ, yₛ) of the point s and the number of pixels (W×H) of the captured image 3 from the captured image 3. Furthermore, the control unit 11 is able to acquire a depth Ds of the point s by referring to the depth information. The control unit 11 is able to calculate the angles γₛ and βₛ of the point s by using this information. Specifically, the angle per pixel in the vertical direction of the captured image 3 can be approximated to a value that is shown in the following equation 1. The control unit 11 is thereby able to calculate the angles γₛ and βₛ of the point s, based on the relational

equations that are shown in the following equations 2 and 3.

$$\begin{matrix}\frac{{V}_{y}}{H} & ( 1 ) \\ {γ}_{s} = \frac{{V}_{y}}{H} \times {y}_{s} & ( 2 ) \\ {β}_{s} = 90 - α - {γ}_{s} & ( 3 )\end{matrix}$$

The control unit 11 is then able to derive the value of Ls, by applying the calculated γₛ and the depth Ds of the point s to the following relational equation 4, Also, the control unit 11 is able to calculate a height hs of the point s within real space by applying the calculated Ls and βₛ to the following relational equation 5.

$$\begin{matrix}{L}_{s} = \frac{{D}_{s}}{cos {γ}_{s}} & ( 4 ) \\ {h}_{s} = {L}_{s} \times cos {β}_{s} & ( 5 )\end{matrix}$$

Accordingly, the control unit 11, by referring to the depth for each pixel that is indicated by the depth information, is able to specify the height within real space of the target appearing in that pixel. In other words, the control unit 11, by referring to the depth for each pixel that is indicated by the depth information, is able to specify the region capturing the target that is located at the height designated based on the position of the knob 43.

Note that the control unit 11, by referring to the depth for each pixel that is indicated by the depth information, is able to specify not only the height hs within real space of the target appearing in that pixel but also the position within real space of the target that is captured in that pixel. For example, the control unit 11 is able to calculate the values of the vector S (Sₓ, S_{y}, S_{z}, 1) from the camera 2 to the point s in the camera coordinate system illustrated in FIG. 11, based on the relational equations shown in the following equations 6 to 8. The position of the point s in the coordinate system within the captured image 3 and the position of the point s in the camera coordinate system are thereby exchangeable.

$$\begin{matrix}{S}_{x} = {x}_{s} \times ( {D}_{s} \times tan \frac{{V}_{x}}{2} ) / \frac{W}{2} & ( 6 ) \\ {S}_{y} = {y}_{s} \times ( {D}_{s} \times tan \frac{{V}_{y}}{2} ) / \frac{H}{2} & ( 7 ) \\ {S}_{z} = {D}_{s} & ( 8 )\end{matrix}$$

Next, the relationship between the height designated based on the position of the knob 43 and the region clearly

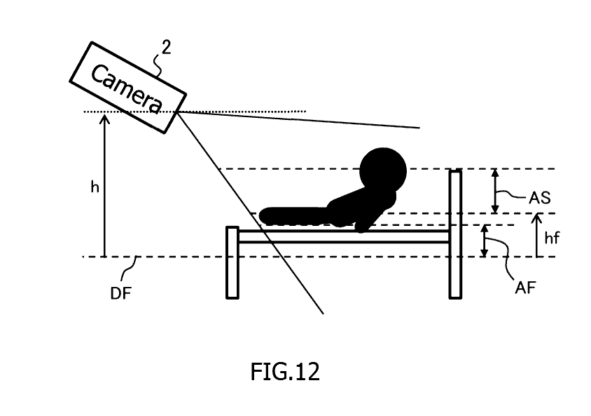

indicated on the captured image 3 will be described using FIG. 12. FIG. 12 schematically illustrates the relationship between a plane (hereinafter, also referred to as the “designated plane”) DF at the height designated based on the position of the knob 43 and the image capturing range of the camera 2. Note that FIG. 12 illustrates a situation in which the camera 2 is viewed from the side, similarly to FIG. 1, and the up-down direction in FIG. 12 corresponds to the height direction of the bed, and also corresponds to the vertical direction within real space.

A height h of a designated plane DF illustrated in FIG. 12 is designated as a result of the user operating the scroll bar 42. Specifically, the position of the knob 43 along the scroll bar 42 corresponds to the height h of the designated plane DF, and the control unit 11 decided the height h of the designated plane DF based on the position of the knob 43 along the scroll bar 42. For example, the user is thereby able to reduce the value of the height h, such that the designated plane DF moves upward within real space, by moving the knob 4 3 upward. On the other hand, the user is able to increase the value of the height h, such that the designated plane DF moves downward within real space, by moving the knob 43 downward.

Here, as described above, the control unit 11 is able to specify the height of the target appearing in each pixel within the captured image 3, based on the depth information. In view of this, the control unit 11, in the case of accepting such designation of the height h by the scroll bar 42, specifies a region, in the captured image 3, showing a target that is located at the height h of this designation, or in other words, a region capturing a target that is located in the designated plane DF. The control unit 11 then functions as the display control unit 25, and clearly indicates, on the captured image 3 that is rendered in the region 41, a portion corresponding to the region capturing the target that is located in the designated plane DF. For example, the control unit 11 clearly indicates a portion corresponding to the region capturing the target that is located in the designated plane DF, by rendering this region in a different display mode to other regions in the captured image 3, as illustrated in FIG. 9.

The method of clearly indicating the region of the target may be set, as appropriate, according to the embodiment. For example, the control unit 11 may clearly indicate the region of the target, by rendering the region of the target in a different display mode from other regions. Here, the display mode utilized for the region of the target need only be a mode that can identify the region of the target, and is specified using color, tone, or the like. To give an example, the control unit 11 renders the captured image 3, which is a monochrome grayscale image, in the region 41. In response to this, the control unit 11 may clearly indicate, on the captured image 3, the region capturing the target that is located at the height of the designated plane DF, by rendering the region capturing the target that is located at the height of this designated plane DF in red. Note that, in order to make the designated plane DF easier to see in the captured image 3, the designated plane DF may have predetermined width (thickness) in the vertical direction.

In this way, in this step S103, the information processing device 1 according to the present embodiment, when accepting designation of the height h by the scroll bar 42, clearly indicates, on the captured image 3, the region capturing the target that is located at the height h. The user sets the height of the bed upper surface with reference to the region that is located at the height of the designated plane DF that is clearly indicated. Specifically, the user sets the height of the bed upper surface, by adjusting the position of the knob 43, such that the designated plane DF coincides with the bed upper surface. That is, the user is able to set the height of the bed upper surface, while grasping the designated height h visually on the captured image 3. In the present embodiment, even a user who has poor knowledge of the watching system is thereby able to easily set the height of the bed upper surface.

Also, in the present embodiment, the upper surface of the bed is employed as the reference plane of the bed. In the case where capturing the behavior in bed of the person being watched over with the camera 2, the upper surface of the bed is a place that is readily appears in the captured image 3 that is acquired by the camera 2. Thus, the bed upper surface tends to occupy a large part of the region of the captured image 3 showing the bed, and the designated plane DF can be readily aligned with such a region showing the bed upper surface. Accordingly, setting of the reference plane of the bed can be facilitated by employing the bed upper surface as the reference plane of the bed as in the present embodiment.

Note that the control unit 11 may function as the display control unit 25 and, when accepting designation of the height h by the scroll bar 42, clearly Indicate, on the captured image 3 that is rendered in the region 41, the region capturing the target that is located in a predetermined range AF upward in the height direction of the bed from the designated plane DF. The region of the range AF is clearly indicated so as to be distinguishable from other regions including the region of the designated plane DF, by being rendered in a different display mode from the other regions, as illustrated in FIG. 9.

Here, the display mode of the region of the designated plane DF corresponds to a “first display mode” of the present invention, and the display mode of the region of range AF corresponds to a “second display mode” of the present invention. Also, the distance in the height direction of the bed that defines the range AF corresponds to a “first predetermined distance” of the present invention. For example, the control unit 11 may clearly indicate the region capturing the target that is located in the range AF on the captured image 3, which is a monochrome grayscale image, in blue.

The user thereby becomes able to visually grasp, on the captured image 3, the region of the target that is located in the predetermined range AF on the upper side of the designated plane DF, in addition to the region that is located at the height of the designated plane DF. Thus, the state within real space of the subject appearing in the captured image 3 is readily grasped. Also, since the user is able to utilize the region of the range AF as an indicator when aligning the designated plane DF with the bed upper surface, setting of the height of the bed upper surface is facilitated.

Note that the distance in the height direction of the bed that defines range AF may be set to the height of the rails of the bed. This height of the rails of the bed may be acquired as a set value set in advance, or may be acquired as an input value from the user. In the case where the range AF is set in this way, the region of the range AF will be a region indicating the region of the rails of the bed, when the designated plane DF is appropriately set to the bed upper surface. In other words, if becomes possible for the user to align the designated plane DF with the bed upper surface, by aligning the region of the range AF with the region of the rails of the bed. Accordingly, setting of the height of the bed upper surface is facilitated, since it becomes possible to utilize the region showing the rails of the bed as an indicator when designating the bed upper surface on the captured image 3.

Also, as will be discussed later, the information processing device 1 detects the person being watched over sitting up in bed, by determining whether the target appearing in a foreground region exists in a position, within real space, that is a predetermined distance hf or more above the bed upper surface set by the designated plane DF. In view of this, the control unit 11 may function as the display control unit 25, and, when accepting designation of the height h by the scroll bar 42, clearly indicate, on the captured image 3 that is rendered in the region 41, the region capturing the target that is located at a height greater than or equal to the distance hf upward in the height direction of the bed from the designated plane DF.

This region at a height greater than or equal to the distance hf upward in the height direction of the bed from the designated plane DF may be configured to have a limited range (range AS) in the height direction of the bed, as illustrated in FIG. 12. The region of this range AS is clearly indicated so as to be distinguishable from other regions including the region of the designated plane DF and the range AF, by being rendered in a different display mode from the other regions, for example.

Here, the display mode of the region of the range AS corresponds to a “third display mode” of the present invention. Also, the distance hf relating to detection of sitting up corresponds to a “second predetermined distance” of the present invention. For example, the control unit 11 may clearly indicate, on the captured image 3 which is a monochrome grayscale image, the region capturing the target that is located in the range AS in yellow.

The user thereby becomes able to visually grasp the region relating to detection of sitting up on the captured image 3. Thus, it becomes possible to set the height of the bed upper surface so as to be suitable for detection of sitting up.

Note that, in FIG. 12, the distance hf is longer than the distance in the height direction of the bed that, defines the range AF. However, the distance hf need not be limited to such a length, and may be the same as the distance in the height direction of the bed that defines the range AF, or may be shorter than this distance. In the case where the distance hf is shorter than the distance in the height direction of the bed that defines the range AF, a region occurs in which the region of the range AF and the region of the range AS overlap. As the display mode of this overlapping region, the display mode of one of the range AF and the range AS may be employed, or a different display mode from both the range AF and the range AS may be employed.

Also, the control unit 11 may function as the display control unit 25, and, when accepting designation of the height h by the scroll bar 42, clearly indicate, on the captured image 3 that is rendered in the region 41, the region capturing the target that is located upward and the region capturing the target that, is located lower down within real space than the designated plane DF in different display modes. By thus rendering the region on the upper side and the region on the lower side of the designated plane DF in respectively different display modes, it can be made easier to visually grasp the region located at the height of the designated plane DF. Therefore, it can be made easier to recognise the region capturing the target that is located at the height of the designated plane DF on the captured image 3, and designation of the height of the bed upper surface is facilitated.

Returning to FIG. 9, a “back” button 44 for accepting redoing of setting and a “next” button 45 for accepting that, setting of the designated plane DF has been completed are further provided on the screen 40. When the user operates the “back” button 44, the control unit 11 of the information processing device 1 returns the processing to step S101. On the other hand, when a user operates the “next” button 45, the control unit 11 finalizes the height of the bed upper surface that is designated. That is, the control unit 11 stores the height of the designated plane DF that has been designated when the button 45 is operated, and sets the stored height of the designated plane DF as the height of the bed upper surface. The control unit 11 then advances the processing to the next step S104.

### Step S104

Returning to FIG. 6, in step S104, the control unit 11 determines whether behavior other than sitting up in bed is included in one or more types of behavior for defection selected in step S101. In the case where behavior other than sitting up is included in the one or more types of behavior selected in step S101, the control unit 11 advances the processing to the next step S105, and accepts setting of the range of the bed upper surface. On the other hand, in the case where behavior other than sitting up is not included in the one or more types of behavior selected in step S101, or in other words, in the case where the only behavior selected in step S101 is sitting up, the control unit 11 ends setting relating to the position of the bed according to this exemplary operation, and starts processing that relates to behavior detection which will be discussed later.

As described above, in the present embodiment, the types of behavior serving as a target to be detected by the watching system are sitting up, being out of bed, edge sitting, and being over the rails. Of these types of behavior, “sitting up” is behavior that has the possibility of being carried out over a wide range of the bed upper surface. Thus, it is possible for the control unit 11 to detect “sitting up” of the person being watched over with comparatively high accuracy, based on the positional relationship in the height direction of the bed between the person being watched over and the bed, even when the range of the bed upper surface is not set.

On the other hand, “out of bed”, “edge sitting”, and “over the rails” are types of behavior that correspond to “predetermined behavior that is carried out in proximity to or on the outer side of an edge portion of the bed” of the present invention, and are carried out in a comparatively limited range. Thus, it is better to set the range of the bed upper surface such that not only the positional relationship in the height direction of the bed between the person being watched over and the bed but also the positional relationship in the horizontal direction between the person being watched over and the bed can be specified, in order for the control unit 11 to accurately detect these types of behavior. That is, it is better to set the range of the bed upper surface, in the case where any of “out of bed”, “edge sitting” and “over the rails” are selected as behavior to be detected in step S101.

In view of this, in the present embodiment, the control unit 11 determines whether such “predetermined behavior” is included in the one or more types of behavior selected in step S101. In the case where “predetermined behavior” is included in the one or more types of behavior selected in step S101, the control unit 11 then advances the processing to the next step S105, and accepts setting of the range of the bed upper surface. On the other hand, in the case where “predetermined behavior” is not included in the one or more types of behavior selected in step S101, the control unit 11 omits setting of the range of the bed upper surface, and ends setting relating to the position of the bed according to this exemplary operation.

That is, the information processing device 1 according to the present embodiment only accepts setting of the range of the bed upper surface in the case where setting of the range of the bed upper surface is recommended, rather than accepting setting of the range of the bed upper surface in all cases. Thereby, in some cases, setting of the range of the bed upper surface can be omitted, enabling setting relating to the position of the bed to be simplified. Also, a configuration can be adopted to accept setting of the range of the bed upper surface, in the case where setting of the range of the bed upper surface is recommended. Thus, even a user who has poor knowledge of the watching system becomes able to appropriately select setting items relating to the position of the bed, according to the behavior selected to be detected.

Specifically, in the present embodiment, in the case where only “sitting up” is selected as behavior to be detected, setting of the range of the bed upper surface is omitted. On the other hand, in the case where at least one type of behavior out of “out of bed”, “edge sitting” and “over the rails” is selected as behavior to be detected, setting of the range of the bed upper surface (step S105) is accepted.

Note that the behavior included in the above-mentioned “predetermined behavior” may be selected, as appropriate, according to the embodiment. For example, the detection accuracy of “sitting up” may be enhanced by setting the range of the bed upper surface. Thus, “sitting up” may be included in the “predetermined behavior” of the present, invention. Also, for example, “out of bed”, “edge sitting” and “over the rails” can possibly be accurately detected, even when the range of the bed upper surface is not set. Thus, any of “out of bed”, “edge sitting” and “over the rails” may be excluded from the “predetermined behavior”

### Step S105

In step S105, the control unit 11 functions as the setting unit 24, and accepts designation of the position of a reference point of the bed and orientation of the bed. The control unit 11 then sets the range within real space of the bed upper surface, based on the designated position of the reference point and orientation of the bed.

FIG. 13 illustrates a screen 50 that is displayed on the touch panel display 13 when accepting setting of the range of the bed upper surface. The control unit 11 displays the screen 50 on the touch panel display 13, in order to accept designation, of the range of the bed upper surface in step S105. The screen 50 includes a region 51 in which the captured image 3 that is obtained from the camera 2 is rendered, a marker 52 for designating a reference point, and a scroll bar 53 for designating the orientation of the bed.

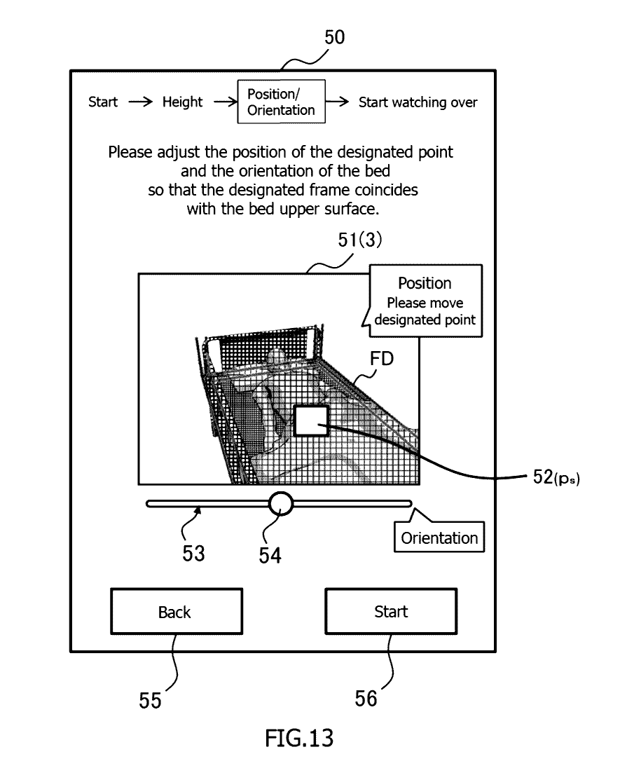

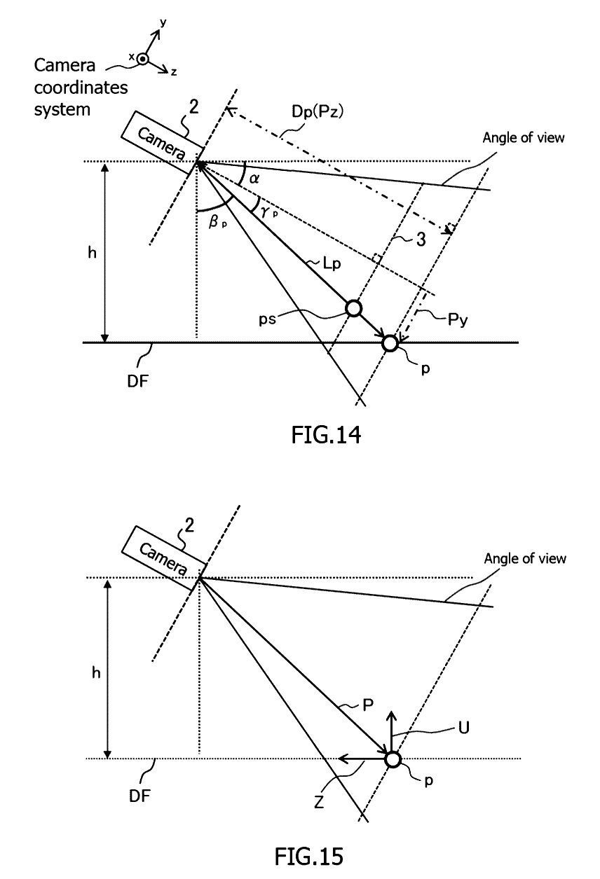

In this step S105, the user designates the position of the reference point on the bed upper surface, by operating the marker 52 on the captured image 3 that is rendered in the region 51. Also, the user operates a knob 54 of the scroll bar 53 to designate the orientation of the bed. The control unit 11 specifies the range of the bed upper surface, based on the position of the reference point and the orientation of the bed that are thus designated. The respective processing will be described using FIGS. 14 to 17.

First, the position of a reference point p that is designated by the marker 52 will be described using FIG. 14. FIG. 14 illustrates the positional relationship between a designated point ps on the captured image 3 and the reference point p of the bed upper surface. The designated point ps indicates the position of the marker 52 on the captured image 3. Also, the designated plane DF illustrated in FIG. 14 indicates a plane that is located at the height h on the bed upper surface set in step S103. In this case, the control unit 11 is able to specify the reference point p that is designated by the marker 52 as an intersection between the designated plane DF and a straight line connecting the camera 2 and the designated point pₛ.

Here, the coordinates of the designated point pₛ on the captured image 3 are given as (xₚ, yₚ). Also, the angle between the line segment connecting the camera 2 and the designated point pₛ and a line segment indicating the vertical direction within real space is given as βₚ, and the angle between the line segment connecting the camera 2 and the designated point pₛ and a line segment indicating the image capturing direction of the camera 2 is given as γₚ. Furthermore, the length of a line segment connecting the reference point p and the camera 2 as viewed from the lateral direction is given as Lₚ, and the depth from the camera 2 to the reference point p is given as Dₚ.

At this time, the control unit 11 is able to acquire information indicating the angle of view (Vₓ, V_{y}) of the camera 2 and the pitch angle α, similarly to step S103. Also, the control unit 11 is able to acquire the coordinates (xₚ, yₚ) of the designated point pₛ on the captured image 3 and the number of pixels (W×H) of the captured image 3. Furthermore, the control unit 11 is able to acquire information indicating the height h set in step S103. The control unit 11 is able to calculate a depth Dₚ from the camera 2 to the reference point p, by applying these values to the relational equations shown by the following equations 9 to 11, similarly to step S103.

$$\begin{matrix}{γ}_{p} = \frac{{V}_{y}}{H} \times {y}_{p} & ( 9 ) \\ {β}_{p} = 90 - α - {γ}_{p} & ( 10 ) \\ {D}_{p} = {L}_{p} \times cos {γ}_{p} = \frac{h}{cos {β}_{p}} \times cos {γ}_{p} & ( 11 )\end{matrix}$$

The control unit 11 is then able to derive coordinates P {Pₓ, P_{y}, P_{z}, 1) in the camera coordinate system of the reference point p, by applying the calculated depth Dₚ to the relational equations shown by the following equations 12 to 14 . It thereby becomes possible for the control unit 11 to specify the position within real space of the reference point p that is designated by the marker 52.

$$\begin{matrix}{P}_{x} = {x}_{p} \times ( {D}_{p} \times tan \frac{{V}_{x}}{2} ) / \frac{W}{2} & ( 12 ) \\ {P}_{y} = {y}_{P} \times ( {D}_{p} \times tan \frac{{V}_{y}}{2} ) / \frac{H}{2} & ( 13 ) \\ {P}_{z} = {D}_{p} & ( 14 )\end{matrix}$$

Note that FIG. 14 illustrates the positional relationship between the designated point pₛ on the captured image 3 and the reference point p of the bed upper surface in the case where the target appearing at the designated point pₛ exists at a higher position than the bed upper surface set in step S103. In the case where the target appearing at the designated point pₛ is located at the height of the bed upper surface set in step S103, the designated point pₛ and the reference point pₛ will be at the same position within real space.

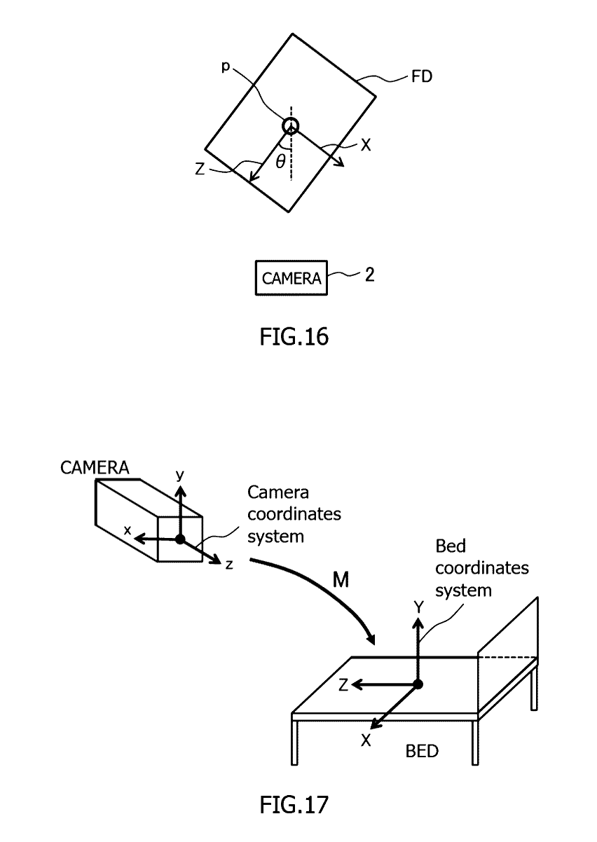

Next, the range of the bed upper surface that is specified based on an orientation 9 of the bed that is designated by the scroll bar 53 and the reference point, p will be described using FIGS. 15 and 16. FIG. 15 illustrates the positional relationship between the camera 2 and the reference point, p in the case where the camera 2 is viewed from the side. Also, FIG. 16 illustrates the positional relationship between the camera 2 and the reference point p in the case where the camera 2 is viewed from above.

The reference point p of the bed upper surface is a point serving as a reference for specifying the range of the bed upper surface, and is set so as to correspond to a predetermined position on the bed upper surface. This predetermined position to which the reference point p is corresponded is not particularly limited, and may be set, as appropriate, according to the embodiment. In the present embodiment, the reference point p is set so as to correspond to the center of the bed upper surface.

In contrast, the orientation θ of the bed according to the present embodiment is represented by the inclination of the bed in the longitudinal direction with respect to the image capturing direction of the camera 2, as illustrated in FIG. 16, and is designated based on the position of the knob 54 along the scroll bar 53. A vector Z illustrated in FIG. 16 indicates the orientation of the bed. When the user moves the knob 54 of the scroll bar 53 leftward on the screen 50, the vector Z rotates in the clockwise direction about the reference point p, or in other words, changes in a direction in which the value of the orientation θ of the bed increases. On the other hand, when the user moves the knob 54 of the scroll bar 53 rightward, the vector Z rotates in the counterclockwise direction about the reference point p, or in other words, changes in a direction in which the value of the orientation θ of the bed decreases.

In other words, the reference point p indicates the position of the center of the bed, and the orientation θ of the bed indicates the degree of horizontal rotation around the center of the bed. Thus, when the orientation Q and the position of the reference point p of the bed are designated, the control unit 11 is able to specify the position and the orientation within real space of a frame FD indicating the range of a virtual bed upper surface, as illustrated in FIG. 16, based on the designated position of the reference point p and orientation θ of the bed.

Note that the size of the frame FD of the bed is set to correspond to the size of the bed. The size of the bed is, for example, defined by the height (vertical length), lateral width (length in the short direction), and longitudinal width (length in the longitudinal direction) of the bed. The lateral width of the bed corresponds to the length of the headboard and the footboard. Also, the longitudinal width of the bed corresponds to the length of the side frame. The size of the bed is often determined in advance according to the watching environment. The control unit 11 may acquire the size of such a bed as a set value set in advance, as a value input by a user, or by being selected from a plurality of set values set in advance.

The frame FD of the virtual bed indicates the range of the bed upper surface that is set based on the position of the reference point p and the orientation θ of the bed that have been designated. In view of this, the control unit 11 may function as the display control unit 25, and render the frame FD that is specified based on the designated position of the reference point p and orientation θ of the bed within the captured image 3. The user thereby becomes able to set the range of the bed upper surface, while checking with the frame FD of the virtual bed that is rendered within the captured image 3. Thus, the possibility of the user making an error in setting of the range of the bed upper surface can be reduced. Note that the frame FD of this virtual bed may also include rails of the virtual bed. It is thereby further possible for the frame FD of this virtual bed to be easily grasped by the user.

Accordingly, in the present embodiment, the user is able to set the reference point p to an appropriate position, by aligning the marker 52 with the center of the bed upper surface appearing in the captured image 3. Also, the user is able to appropriately set the orientation θ of the bed, by deciding the position of the knob 54 such that the frame FD of the virtual bed overlaps with the periphery of the upper surface of the bed appearing in the captured image 3. Mote that the method of rendering the frame FD of the virtual bed within the captured image 3 may be set, as appropriate, according to the embodiment. For example, a method of utilizing projective transformation described below may be used.

Here, in order to make it easy to grasp the position of the frame FD of the bed and the position of the detection region, which will be discussed later, the control unit 11 may utilize a bed coordinate system that is referenced on the bed. The bed coordinate system is a coordinate system in which, the reference point p of the bed upper surface is given as the origin, the width direction of the bed is given as the x-axis, the height direction of the bed is given as the y-axis, and the longitudinal direction of the bed as given as the z-axis, for example. With such a coordinate system, it is possible for the control unit 11 to specify the position of the frame FD of the bed, based on the size of the bed. Hereinafter, a method of calculating a projective transformation matrix M that transforms the coordinates of the camera coordinate system into the coordinates of this bed coordinate system will be described.

First, a rotation matrix R that pitches the image capturing direction of the horizontally-oriented camera at an angle α is represented by the following equation 15. The control unit 11 is able to respectively derive the vector Z indicating the orientation of the bed in the camera coordinate system and a vector U indicating upward in the height direction of the bed in the camera coordinate system, as illustrated in FIG. 15, by applying this rotation matrix R to the relational equations shown in the following equations 16 and 17. Note that “*” that is included in the relational equations shown in equations 16 and 17 signifies multiplication of the matrices.

$$\begin{matrix}R = ( \begin{matrix}cos α & 0 & sin α & 0 \\ sin α & cos α & 0 & 0 \\ - sin α & 0 & cos α & 0 \\ 0 & 0 & 0 & 1\end{matrix} ) & ( 15 ) \\ Z = ( \begin{matrix}sin θ & 0 & - cos θ & 0\end{matrix} ) * R & ( 16 ) \\ U = ( \begin{matrix}0 & 1 & 0 & 0\end{matrix} ) * R & ( 17 )\end{matrix}$$

Next, the control unit 11 is able to derive a unit vector X of the bed coordinate system in the width direction of the bed, as illustrated in FIG. 16, by applying the vectors U and Z to the relational equation shown in the following equation 18. Also, the control unit 11 is able to derive a unit vector Y of the bed coordinate system in the height direction of the bed, by applying the vector Z and X to the relational equation shown in the following equation 19. The control unit 11 is then able to derive the projective transformation matrix M that transforms coordinates of the camera coordinate system into coordinates of the bed coordinate system, by applying the coordinates P of the reference point p and the vectors X, Y, and Z in the camera coordinate system to the relational equation shown in the following equation 20. Note that “x” that is included in the relational equations shown in equations 18 and 19 signifies the cross product of the vectors.

$$\begin{matrix}X = \frac{U \times Z}{\vert U \times Z \vert} & ( 18 ) \\ Y = Z \times X & ( 19 ) \\ M = ( \begin{matrix}{X}_{x} & {Y}_{x} & {Z}_{x} & 0 \\ {X}_{y} & {Y}_{y} & {Z}_{y} & 0 \\ {X}_{z} & {Y}_{z} & {Z}_{z} & 0 \\ - P \cdot X & - P \cdot Y & - P \cdot Z & 1\end{matrix} ) & ( 20 )\end{matrix}$$

FIG. 17 illustrates the relationship between the camera coordinate system and the bed coordinate system according to the present embodiment. As illustrated in FIG. 17, the projective transformation matrix M that is calculated is able to transform coordinates of the camera coordinate system into coordinates of the bed coordinate system. Accordingly, if the inverse matrix of the projective transformation matrix M is utilized, coordinates of the bed coordinate system can be transformed into coordinates of the camera coordinate system. In other words, if becomes possible to mutually transform coordinates of the camera coordinate system and coordinates of the bed coordinate system, by utilizing the projective transformation matrix M. Here, as described above, coordinates of the camera coordinate system and coordinates within the captured image 3 can be mutually transformed. Thus, coordinates of the bed coordinate system and coordinates within the captured image 3 can be mutually transformed at this time.

Here, as described above, in the case where the size of the bed has been specified, the control unit 11 is able to specify the position of the frame FD of the virtual bed in the bed coordinate system. In other words, the control unit 11 is able to specify the coordinates of the frame FD of the virtual bed in the bed coordinate system. In view of this, the control unit 11 inverse transforms the coordinates of the frame FD in the bed coordinate system, into the coordinates of the frame FD in the camera coordinate system utilizing the projective transformation matrix M.

Also, the relationship between coordinates of the camera coordinate system and coordinates in the captured image is represented by the relational equations shown in the above equations 6 to 8. Thus, the control unit 11 is able to specify the position of the frame FD that is rendered within the captured image 3 from the coordinates of the frame FD in the camera coordinate system, based on the relational equations shown in the above equations 6 to 8. In other words, the control unit. 11 is able to specify the position of the frame FD of the virtual bed in each coordinate system, based on the projective transformation matrix M and information indicating the size of the bed. In this way, the control unit 11 may render the frame FD of the virtual bed in the captured image 3, as illustrated in FIG. 13.

Returning to FIG. 13, a “back” button 55 for accepting redoing of setting and a “start” button 56 for completing setting and starting watching over are further provided on the screen 50. When the user operates the “back” button 55, the control unit 11 returns the processing to step S103.

On the other hand, when the user operates the “start” button 56, the control unit 11 finalizes the position of the reference point p and the orientation θ of the bed. That is, the control unit 11 sets, as the range of the bed upper surface, the range of the frame FD of the bed specified based on the position of the reference point p and the orientation θ of the bed that had been designated when the button 56 was operated. The control unit 11 then advances the processing to the next step S106.

Thus, in the present embodiment, the range of the bed upper surface can be set by specifying the position of the reference point p and the orientation θ of the bed. For example, the entire bed is not necessarily included in the captured image 3, as illustrated in FIG. 13. Thus, in a system that needs to specify the four corners of the bed, for example, in order to set the range of the bed upper surface, it may riot be possible to set the range of the bed upper surface. However, in the present embodiment, only one point {reference point p) designating a position is needed in order to set the range of the bed upper surface. In the present embodiment, the degree of freedom of the installation position of the camera 2 can thereby be enhanced, and application of the watching system to the watching environment can be facilitated.

Also, in the present embodiment, the center of the bed upper surface is employed as the predetermined position to which the reference point p is corresponded. The center of the bed upper surface is a place that readily appears in the captured image 3, whatever direction the bed is captured from. Thus, the degree of freedom of the installation position of the camera 2 can be further enhanced, by employing the center of the bed upper surface as the predetermined position to which the reference point p is corresponded.

When the degree of freedom of the installation position of the camera 2 increases, however, the selection range for arranging the camera 2 widens, and it is possible that arranging the camera 2 may conversely become difficult for the user. In contrast, the present embodiment facilitates arrangement of the camera 2 by instructing the user as to arrangement of the camera 2 while displaying candidate arrangement positions of the camera 2 on the touch panel display 13, and has thus solved such a problem.

Note that the method of storing the range of the bed upper surface may be set, as appropriate, according to the embodiment. As described above, using the projective transformation matrix M that transforms from the camera coordinate system into the bed coordinate system and information indicating the size of bed, the control unit 11 is able to specify the position of the frame FD of the bed. Thus, the information processing device 1 may store, as information indicating the range of the bed upper surface set in step S105, information indicating the size of the bed and the projective transformation matrix M that is calculated based on the position of the reference point p and the orientation 9 of the bed that had been designated when the button 56 was operated.

### Steps S106 to S108

In step S106, the control unit 11 functions as the setting unit 24, and determines whether the detection region of the “predetermined behavior” selected in step S101 appears in the captured image 3. In the case where it is determined that the detection region of the “predetermined behavior” selected in step S101 does not appear in the captured image 3, the control unit 11 then advances the processing to the next step S107. On the other hand, in the case where it is determined that the detection region of the “predetermined behavior” selected in step S101 does appears in the captured image 3, the control unit 11 ends setting relating to the position of the bed according to this exemplary operation, and start processing relating to behavior detection which will be discussed later.

In step S107, the control unit 11 functions as the setting unit 24, and outputs a warning message indicating that there is a possibility that detection of the “predetermined behavior” selected in step S101 cannot be performed normally on the touch panel display 13 or the like. Information indicating the “predetermined behavior” that possibly cannot be detected normally and the location of the detection region that does not appear in the captured image 3 may be included in a warning message.

The control unit 11 then, together with or after this warning message, accepts selection of whether to perform a resetting before performing watching over of the person being watched over, and advances the processing to the next step S108. In step S108, the control unit 11 determines whether to perform resetting based on the selection by the user. In the case where the user selected to perform resetting, the control unit 11 returns the processing to step S105. On the other hand, in the case where the user selected not to perform resetting, the control unit 11 ends setting relating to the position of the bed according to this exemplary operation, and starts processing relating to behavior detection which will be discussed later.

Note that the detection region of “predetermined behavior” is, as will be discussed later, a region that is specified based on the predetermined condition for detecting the “predetermined behavior” and the range of the bed upper surface set in step S105. That is, the detection region of this “predetermined behavior” is a region defining the position of the foreground region in which the person being watched over appears when carrying out the “predetermined behavior”, Thus, the control unit 11 is able to detect the respective types of behavior of the person being watched over, by determining whether the target appearing in the foreground region is included in this detection region.

Thus, in the case where the detection region does not appear within the captured image 3, the watching system according to the present embodiment may possibly be unable to appropriately detect the target behavior of the person being watched over. In view of this, the information processing device 1 according to the present embodiment determines, using step S106, whether there is a possibility that such target behavior of the person being watched over cannot be appropriately detected. The information processing device 1 is then able to inform a user that there is a possibility that the behavior of the target cannot be appropriately detected, by outputting a warning message using step S107, if there is such a possibility. Thus, in the present embodiment, erroneous setting of the watching system can be reduced.

Note that the method of determining whether the detection region appears within the captured image 3 may be set, as appropriate, according to the embodiment. For example, the control unit may specify whether the defection region appears within the captured image 3, by determining whether a predetermined point of the defection region appears within the captured image 3.

### Other Matters

Note that the control unit 11 may function as the non-completion notification unit 28, and, in the case where setting relating to the position of the bed according to this exemplary operation is not completed within a predetermined period of time after starting the processing of step S101, may perform notification for informing that the setting relating to the position of the bed has not been completed. The watching system from being left with setting relating to the position of the bed partially completed can be prevented.

Here, the predetermined period of time serving as a guide for notifying that setting relating to the position of the bed is uncompleted may be determined in advance as a set value, may be determined using a value input by a user, or may be determined by being selected from a plurality of set values. Also, the method of performing notification for informing that such setting is uncompleted may be set, as appropriate, according to the embodiment.

For example, the control unit 11 performs this setting non-completion notification, in cooperation with equipment installed in the facility such as a nurse call that is connected to the information processing device 1. For example, the control unit 11 may control the nurse call connected via the external interface 15 and perform a call by the nurse call, as notification for informing that setting relating to the position of the bed in uncompleted. It thereby becomes possible to appropriately inform the user who watches over the behavior of the person being watched over that setting of watching system is uncompleted.

Also, for example, the control unit 11 may perform notification that setting is uncompleted, by outputting audio from the speaker 14 that is connected to the information processing device 1. In the case where this speaker 14 is disposed in the vicinity of the bed, it is possible, by performing such notification with the speaker 14, to inform a person in the vicinity of the place where watching over is performed that setting of the watching system is uncompleted. This person in the vicinity of the place where watching over is performed may include the person being watched over. It is thereby possible to also notify the actual person being watched over that setting of watching system is uncompleted,

Also, for example, the control unit 11 may cause a screen for informing that setting is uncompleted to be displayed on the touch panel display 13. Also, for example, the control unit 11 may perform such notification utilizing e-mail. In this case, for example, an e-mail address of a user terminal serving as the notification destination is registered in advance in the storage unit 12, and the control unit 11 performs notification for informing that setting is uncompleted, utilizing this e-mail address registered in advance,

### Behavior Detection of Person Being Watched Over

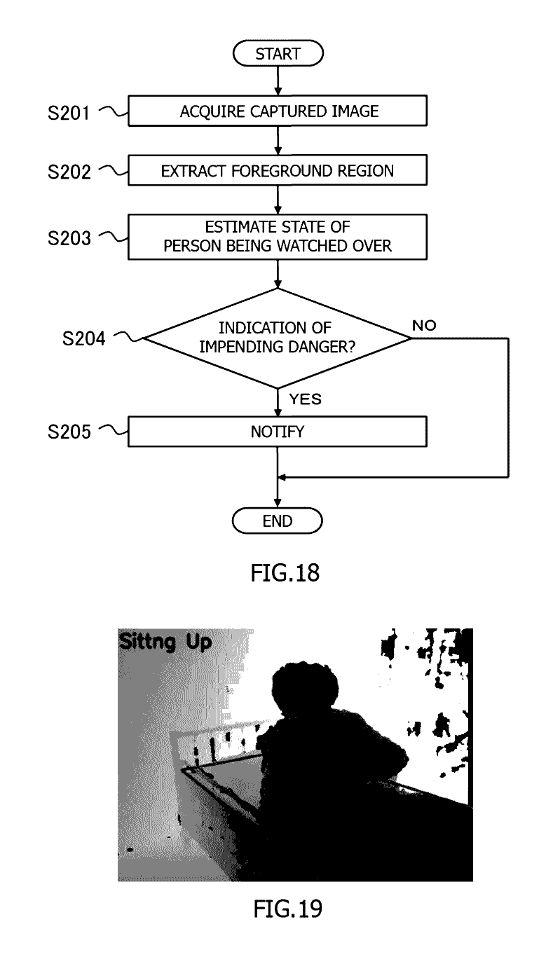

Next, the processing procedure of behavior detection of the person being watched over by the information processing device 1 will be described using FIG. 18. FIG. 18 illustrates the processing procedure of behavior detection of the person being watched over by the information processing device 1. This processing procedure relating to behavior detection is merely an example, and the respective processing may be modified to the full extent possible. Also, with regard to the processing procedure described below, steps can be omitted, replaced or added, as appropriate, according to the embodiment.

### Step S201

In step S201, the control unit 11 function as the image acquisition unit 21, and acquires the captured image 3 captured by the camera 2 installed in order to watch over the behavior in bed of the person being watched over. In the present embodiment, since the camera 2 has a depth sensor, depth information indicating the depth for each pixel is included in the captured image 3 that is acquired.

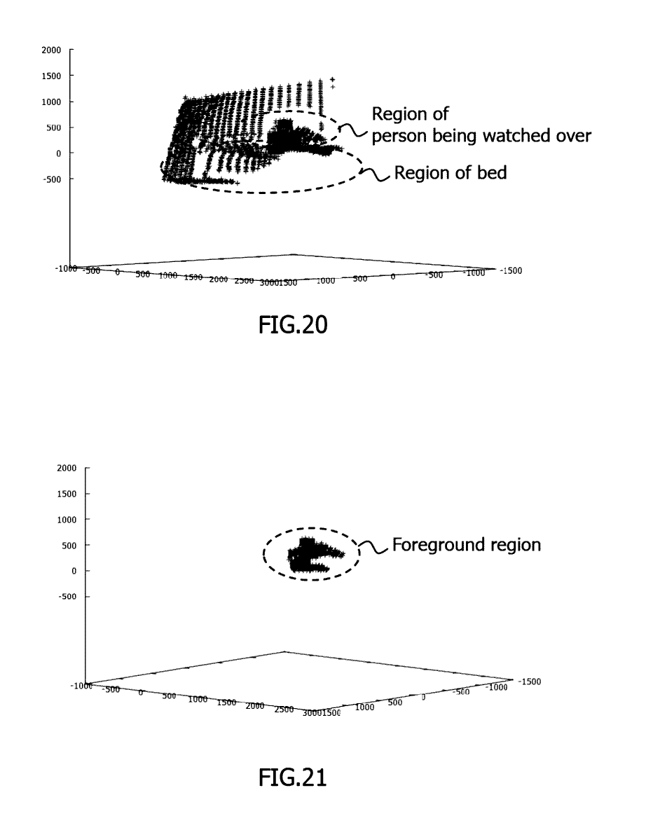

Here, the captured image 3 that the control unit 11 acquires will be described using FIGS. 19 and 20. FIG. 19 illustrates the captured image 3 that is acquired by the control unit 11. The gray value of each pixel of the captured image 3 illustrated in FIG. 19 is determined according to the depth for each pixel, similarly to FIG. 2. That is, the gray value (pixel value) of each pixel corresponds to the depth of the target appearing in that pixel.

The control unit 11 is able to specify the position in real space of the target that appears in each pixel, based on the depth information, as described above. That is, the control unit 11 is able to specify, from the position (two-dimensional information) and depth for each pixel within the captured image 3, the position in three-dimensional space (real space) of the subject appearing within that pixel. For example, the state in real space of the subject appearing in the captured image 3 illustrated in FIG. 19 is illustrated in the following FIG. 20.

FIG. 20 illustrates the three-dimensional distribution of positions of the subject within the image capturing range that is specified based on the depth information that is included in the captured image 3. The three-dimensional distribution illustrated in FIG. 20 can be created by plotting each pixel within three-dimensional space with the position and depth within the captured image 3. In other words, the control unit 11 is able to recognize the state within real space of the subject appearing in the captured image 3, in a manner such as the three-dimensional distribution illustrated in FIG. 20.

Note that the information processing device 1 according to the present embodiment is utilized in order to watch over inpatients or facility residents in a medical facility or a nursing facility. In view of this, the control unit 11 may acquire the captured image 3 in synchronization with the video signal of the camera 2, so as to be able to watch over the behavior of inpatients or facility residents in real time. The control unit 11 may then immediately execute the processing of steps S202 to S205 discussed later on the captured image 3 that is acquired. The information processing device 1 realizes real-time image processing, by continuously executing such an operation without interruption, enabling the behavior of inpatients or facility residents to be watched over in real time.

### Step S202

Returning to FIG. 18, at step S202, the control unit 11 functions as the foreground extraction unit 22, and extracts a foreground region of the captured image 3, from the difference between a background image set as the background of the captured image 3 acquired at step S201 and the captured image 3. Here, the background image is data that is utilized in order to extract the foreground region, and is set to include the depth of a target serving as the background. The method of creating the background image may be set, as appropriate, according to the embodiment. For example, the control unit 11 may create the background image by calculating an average captured image for several frames that are obtained when watching over of the person being watched over is started. At this time, a background image including depth information is created as a result of the average captured image being calculated to also include depth information.

FIG. 21 illustrates the three-dimensional distribution of a foreground region, of the subject illustrated in FIGS. 19 and 20, that is extracted from the captured image 3. Specifically, FIG. 21 illustrates the three-dimensional distribution of the foreground region that is extracted when the person being watched over sits up in bed. The foreground region that, is extracted utilizing a background image such as described above appears in a different position from the state within real space shown in the background image. Thus, in the case where the person being watched over has moved in bed, the region in which the moving part of the person being watched over appears is extracted as this foreground region. For example, in FIG. 21, since the person being watched over has moved to enhance his or her upper body (sit up) in bed, the region in which the upper body of the person being watched over appears is extracted as the foreground region. The control unit 11 determines the movement of the person being watched over, using such a foreground region,

Note that, in this step S202, the method by which the control unit 11 extracts the foreground region need not be limited to a method such as the above, and the background and the foreground may be separated using a background difference method. As the background difference method, for example, a method of separating the background and the foreground from the difference between a background image such as described above and an input image (captured image 3), a method of separating the background and the foreground using three different images, and a method of separating the background and the foreground by applying a statistical model can be given. The method of extracting the foreground region is not particularly limited, and may be selected, as appropriate, according to the embodiment,

### Step S203

Returning to FIG. 18, in step S203, the control unit 11 functions as the behavior detection unit 23, and determines whether the positional relationship between the target appearing in the foreground region and the bed upper surface satisfies a predetermined condition, based on the depths of the pixels within the foreground region extracted in step S102. The control unit 11 then detects the behavior that the person being watched over is carrying out, out the behavior selected to be watched for, based on the result of this determination.

Here, in the case where “sitting up” is selected as behavior to be detected,, in the setting processing about the position of the bed, setting of the range of the bed upper surface is omitted, and only the height of the bed upper surface is set. In view of this, the control unit 11 detects the person being watched over sitting up, by determining whether the target appearing in the foreground region exists at a position higher than the set bed upper surface by a predetermined distance or more within real space.

On the other hand, in the case where at least one of “out of bed”, “edge sitting” and “over the rails” is selected as behavior to be detected, the range within real space of the bed upper surface is set as a reference for detecting the behavior of the person being watched over. In view of this, the control unit 11 detects the behavior selected to be watched for, by determining whether the positional relationship within real space between the set bed upper surface and the target appearing in the foreground region satisfies a predetermined condition.

That is, the control unit 11, in all cases, detects the behavior of the person being watched over, based on the positional relationship within real space between the target appearing in the foreground region and the bed upper surface. Thus, the predetermined condition for detecting the behavior of the person being watched over can correspond to a condition for determining whether the target appearing in the foreground region is included in a predetermined region that is set with the bed upper surface as a reference. This predetermined, region corresponds to the abovementioned detection region. In view of this, hereinafter, for convenience of description, a method of detecting the behavior of the person being watched over based on the relationship between this detection region and the foreground region will be described.

The method of detecting the behavior of the person being watched over is, however, not limited to a method that is based on this detection region, and may be set, as appropriate, according to the embodiment. Also, the method of determining whether the target appearing in a foreground region is included in the detection region may be set, as appropriate, according to the embodiment. For example, it may be determined whether the target appearing in the foreground region is included in the detection region, by evaluating whether a foreground region of a number of pixels greater than or equal to a threshold appears in the detection region. In the present embodiment, “sitting up”, “out of bed”, “edge sitting” and “over the rails” are illustrated as behavior to be detected. The control unit 11 detects these types of behavior as follows.

### (1) Sitting Up

In the present, embodiment, if “sitting up” is selected as the behavior to be detected in step S101, the person being watched over “sitting up” is the determination target, of this step S203. In detection of sitting up, the height of the bed upper surface set in step S103 is used. When setting of the height of the bed upper surface in step S103 is completed, the control unit 11 specifies the detection region for detecting sitting up, based on the height of the set bed upper surface.

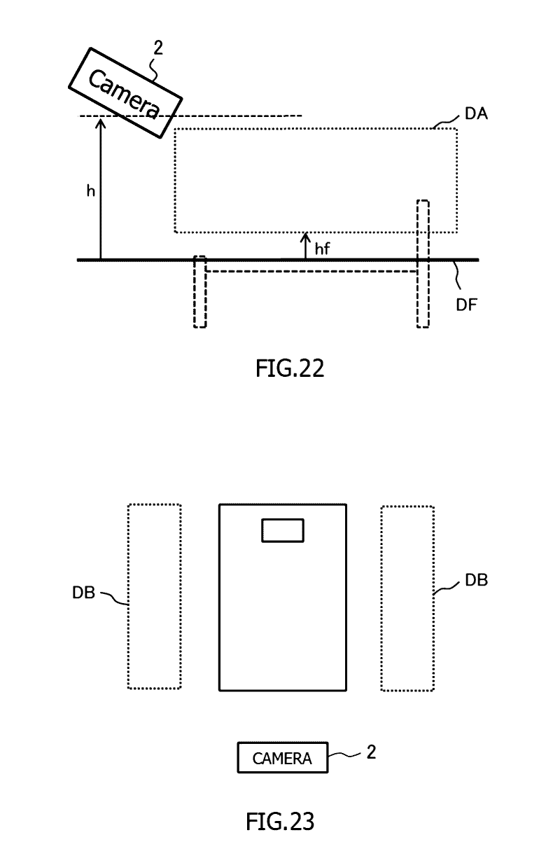

FIG. 22 schematically illustrates a detection, region DA for detecting sitting up. The detection region DA is, for example, set to a position that is greater than, or equal to the distance hf upward in the height direction of the bed from the designated plane (bed upper surface) DF designated in step S103, as illustrated in FIG. 22. This distance hf corresponds to a “second predetermined distance” of the present invention. The range of the detection region DA is not particularly limited, and may be set, as appropriate, according to the embodiment. The control unit 11 may detect the person being watched over sitting up in bed, in the case where it is determined that the target appearing in the foreground region corresponding to a number of pixels greater than or equal to a threshold is included in the detection region DA.

### (2) Out of Bed

In the case where “out of bed” is selected as behavior to be detected in step S101, the person being watched over being “out of bed” is the determination target of this step S203. The range of the bed upper surface set in step S105 is used in detection of being out of bed. When setting of the range of the bed upper surface in step S105 is completed, the control unit 11 is able to specify a detection region for detecting being out of bed, based on the set range of the bed upper surface.

FIG. 23 schematically illustrates a detection region DB for detecting being out of bed. In the case where the person being watched over has gotten out of bed, it is assumed that the foreground region will appear in a position away from the side frame of the bed. In view of this, the detection region DB may be set to a position away from the side frame of the bed based on the range of the bed upper surface specified in step S105, as illustrated in FIG. 23. The range of the detection region DB may be set, as appropriate, according to the embodiment, similarly to the detection region DA. The control unit 11 may detect the person being watched over being out of bed, in the case where it is determined that the target appearing in the foreground region corresponding to a number of pixels greater than or equal to a threshold is included in the detection region DB.

### (3) Edge Sitting

In the case where “edge sitting” is selected as behavior to be detected in step S101, the person being watched over “edge sitting” is the determination target of this step S203. The range of the bed upper surface set in step S105 is used in detection of edge sitting, similarly to detection of being out of bed. When setting of the range of the bed upper surface in step S105 is completed, the control unit 11 is able to specify the detection region for detecting edge sitting, based on the set range of the bed upper surface.

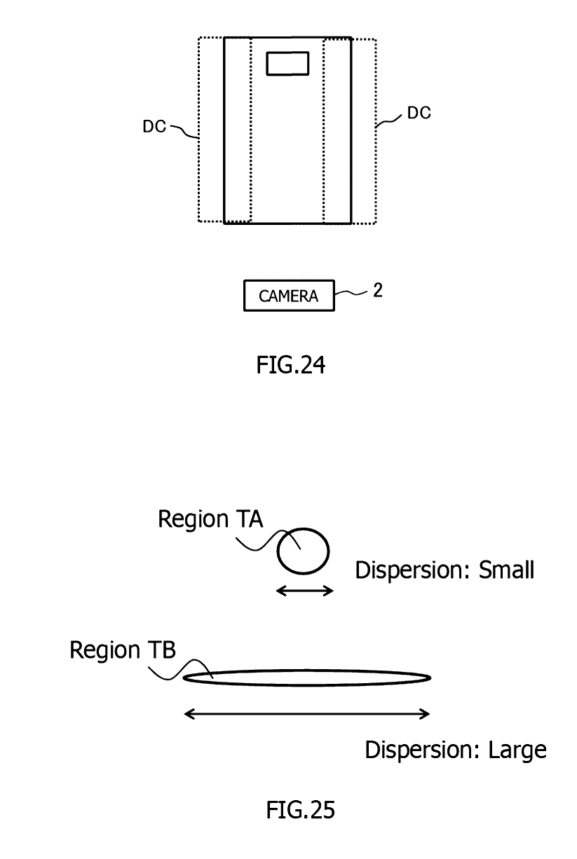

FIG. 24 schematically illustrates a detection region DC for detecting edge sitting. In the case where the person being watched over sits upright on the bed, it is assumed that the foreground region will appear on the periphery of the side frame of the bed and also from above to below the bed. In view of this, the detection region DC may be set on the periphery of the side frame of the bed and also from above to below the bed, as illustrated in FIG. 24. The control unit 11 may detect the person being watched over edge sifting on the bed, in the case where it is determined that the target appearing in the foreground region corresponding to a number of pixels greater than or equal to a threshold is included in the detection region DC.

### (4) Over the Rails

In the case where “over the rails” is selected as behavior to be detected in step S101, the person being watched over being “over the rails” is the determination target of this step S203. The range of the bed upper surface set in step S105 is used in detection of over the rails, similarly to detection of being out of bed and edge sitting. When setting of the range of the bed upper surface in step S105 is completed, the control unit 11 is able to specify the detection region for detecting being over the rails, based on the set range of the bed upper surface.

Here, in the case where the person being watched over is positioned over the rails, it is assumed that the foreground region will appear on the periphery of the side frame of the bed and also above the bed. In view of this, the detection region for detecting being over the rails may be set to the periphery of the side frame of the bed and also above the bed. The control unit 11 may detect the person being watched over being over the rails, in the case where it is determined that the target appearing in the foreground region corresponding to a number of pixels greater than or equal to a threshold is included in this detection region.

### (5) Other Processing

In this step S203, the control unit 11 performs detection of each type of behavior selected in step S101. That is, the control unit 11 is able to detect the target behavior, in the case where it is determined that the above determination condition of the target behavior is satisfied. On the other hand, in the case where it is determined that the above determination condition of each type of behavior selected in step S101 is not satisfied, the control unit 11 advances the processing to the next step S204, without detecting the behavior of the person being watched over.

Note that, as described above, in step S105, the control unit 11 is able to calculate the projective transformation matrix M that transforms vectors of the camera coordinate system into vectors of the bed coordinate system. Also, the control unit 11 is able to specify coordinates S (Sₓ, S_{y}, S_{z}, 1) in the camera coordinate system of the arbitrary point s within the captured image 3, based on the above equations 6 to 8. In view of this, the control unit 11 may, when detecting the respective types of behavior in (2) to (4), calculate the coordinates in the bed coordinate system of each pixel within the foreground region, utilizing this projective transformation matrix M. The control unit 11 may then determine whether the target appearing in each pixel within, the foreground region is included in the respective detection region, utilizing the coordinates of the calculated bed coordinate system.

Also, the method of detecting the behavior of the person being watched over need not be limited to the above method, and may be set, as appropriate, according to the embodiment. For example, the control unit 11 may calculate an average position of the foreground region, by taking the average position and depth of respective pixels within the captured image 3 that are extracted as the foreground region. The control unit 11 may then detect the behavior of the person being watched over, by determining whether the average position of the foreground region is included in the detection region set as a condition for detecting each type of behavior within real space.

Furthermore, the control unit 11 may specify the part of the body appearing in the foreground region, based on the shape of the foreground region. The foreground region shows the change from the background image. Thus, the part of the body appearing in the foreground region corresponds to the moving part of the person being watched over. Based on this, the control unit 11 may detect the behavior of the person being watched over, based on the positional relationship between the specified body part (moving part) and the bed upper surface. Similarly to this, the control unit 11 may detect the behavior of the person being watched over, by determining whether the part of the body appearing in the foreground region that is included in the detection region for each type of behavior is a predetermined body part.

### Step S204

In step S204, the control unit 11 functions as the danger indication notification unit 27, and determines whether the behavior detected in step S203 is behavior showing an indication that the person being watched over is in impending danger. In the case where the behavior detected in step S203 is behavior showing an indication that the person being watched over is in impending danger, the control unit 11 advances the processing to step S205. On the other hand, in the case where the behavior of the person being watched over is not detected in step S203, or in the case where the behavior detected in step S203 is not behavior showing an indication that the person being watched over is in impending danger, the control unit 11 ends the processing relating to this exemplary operation.

Behavior that is set as behavior showing an indication that the person being watched over is in impending danger may be selected, as appropriate, according to the embodiment. For example, as behavior that may possibly result in the person being watched over rolling or falling, assume that edge sitting is set as behavior showing an indication that the person being watched over is in impending danger. In this case, the control unit 11 determines that, when it is detected in step S203 that the person being watched over is edge sitting, the behavior detected in step S203 is behavior showing an indication that the person being watched over is in impending danger,

In the case of determining whether the behavior detected in this step S203 is behavior showing an indication that the person being watched over is in impending danger, the control unit 11 may take into consideration the transition in behavior of the person being watched over. For example, it is assumed that there is a greater chance of the person being watched over rolling or falling when changing from sitting up to edge sitting than when changing from being out of bed to edge sitting. In view of this, the control unit 11 may determine, in step S204, whether the behavior detected in step S203 is behavior showing an indication that the person being watched over is in impending danger in light of the transition in behavior of the person being watched over.

For example, assume that the control unit 11, when periodically detecting the behavior of the person being watched over, detects, in step S203, that the person being watched over has changed to edge sitting, after having detected that the person being watched over is sitting up. At this time, the control unit 11 may determine, in this step S204, that the behavior inferred in step S203 is behavior showing an indication that the person being watched over is in impending danger.

### Step S205

In step S205, the control unit 11 functions as the danger indication notification unit 27, and performs notification for informing that there is an indication that the person being watched over is in impeding danger. The method by which the control unit 11 performs the notification may be set, as appropriate, according to the embodiment, similarly to the setting non-completion notification,

For example, the control unit 11 may, similarly to the setting non-completion notification, perform notification for informing that there is an indication that the person being watched over is in impending danger utilizing a nurse call, or utilizing the speaker 14. Also, the control unit 11 may display notification for informing that there is an indication that the person being watched over is in impending danger on the touch panel display 13, or may perform this notification utilizing an e-mail.

When this notification is completed, the control unit 11 ends the processing relating to this exemplary operation. The information processing device 1 may, however, periodically repeat the processing that is shown in an abovementioned exemplary operation, in the case of periodically detecting the behavior of the person being watched over. The interval for periodically repeating the processing may be set as appropriate. Also, the information processing device 1 may perform the processing shown in the above-mentioned exemplary operation, in response to a request from the user.

As described above, the information processing device 1 according to the present embodiment detects the behavior of the person being watched over, by evaluating the positional relationship within real space between the moving part of the person being watched over and the bed, utilizing a foreground region and the depth of the subject. Thus, according to the present embodiment, behavior inference in real space that is in conformity with the state of the person being watched over is possible.

### 4. Modifications

Although embodiments of the present invention have been described above in detail, the foregoing description is in all respects merely an illustration of the invention. It should also be understood that various improvement and modification can be made without departing from the scope of the invention.

### (1) Utilization of Area

For example, the image of the subject within the captured image 3 becomes smaller, the further the subject is from the camera 2, and the image of the subject within the captured image 3 increases, the closer the subject is to the camera 2. Although the depth of the subject appearing in the captured image 3 is acquired with respect to the surface of that subject, the area of the surface portion of the subject corresponding to each pixel of that captured image 3 does not necessarily coincide among the pixels.

In view of this, the control unit 11, in order to exclude the influence of the nearness or farness of the subject, may, in the above step S203, calculate the area within real space of the portion of the subject appearing in a foreground region that is included in the detection region. The control unit 11 may then detect the behavior of the person being watched over, based on the calculated area.

Note that the area within real space of each pixel within the captured image 3 can be derived as follows, based on the depth for the pixel. The control unit 11 is able to respectively calculate a length w in the lateral direction and a length h in the vertical direction within real space of an arbitrary point s (1 pixel) illustrated in FIGS. 10 and 11, based on the following relational equations 21 and 22.

$$\begin{matrix}w = ( {D}_{s} \times tan \frac{{V}_{x}}{2} ) / \frac{W}{2} & ( 21 ) \\ h = ( {D}_{s} \times tan \frac{{V}_{y}}{2} ) / \frac{H}{2} & ( 22 )\end{matrix}$$

Accordingly, the control unit 11 is able to derive the area within real space of one pixel at a depth Ds, by the square of w, the square of h, or the product of w and h thus calculated. In view of this, the control unit 11, in the above step S203, calculates the total area within real space of those pixels in the foreground region that capture the target that is included in the detection region. The control unit 11 may then detect the behavior in bed of the person being watched over, by determining whether the calculated total area is included within a predetermine range. The accuracy with which the behavior of the person being watched over is detected can thereby be enhanced, by excluding the influence of the nearness or farness of the subject.

Note that this area may change greatly depending on factors such as noise in the depth information and the movement of objects other than the person being watched over. In order to address this, the control unit 11 may utilize the average area for several frames. Also, the control unit 11 may, in the case where the difference between the area of the region in the frame to be processed and the average area of that region for the past several frames before the frame to be processed exceeds a predetermined range, exclude that region from being processed.

(2) Behavior Estimation utilizing Area and Dispersion

In the case of detecting the behavior of the person being watched over utilizing an area such as the above, the range of the area serving as a condition for detecting behavior is set based on a predetermined part of the person being watched over that is assumed to be included in the detection region. This predetermined part may, for example, be the head, the shoulders or the like of the person being watched over. That is, the range of the area serving as a condition for detecting behavior is set, based on the area of a predetermined part of the person being watched over.

With only the area within real space of the target appearing in the foreground region, the control unit 11 is, however, not able to specify the shape of the target appearing in the foreground region. Thus, the control unit 11 may possibly erroneously detect the behavior of the person being watched over for the part of the body of the person being watched over that is included in the detection region. In view of this, the control unit 11 may prevent such erroneous detection, utilizing a dispersion showing the degree of spread within real space.

This dispersion will be described using FIG. 25. FIG. 25 illustrates the relationship between dispersion and the degree of spread of a region. Assume that a region TA and a region TB illustrated in FIG. 25 respectively have the same area. When inferring the behavior of the person being watched over with only areas such as the above, the control unit 11 recognizes the region TA and the region TB as being the same, and thus there is a possibility that the control unit 11 may erroneously detect the behavior of the person being watched over.

However, the spread within real space greatly differs between the region TA and the region TB, as illustrated in FIG. 25 (degree of horizontal spread in FIG. 25). In view of this, the control unit 11, in the above step S203, may calculate the dispersion of those pixels in the foreground region that capture the target included in the detection region. The control unit 11 may then detect the behavior of the person being watched over, based on the determination of whether the calculated dispersion is included in a predetermined range.

Note that, similarly to the example of the above area, the range of the dispersion serving as a condition for detecting behavior is set based on a predetermined part of the person being watched over that is assumed to be included in the detection region. For example, in the case where it is assumed that the predetermined part that is included in the detection region is the head, the value of the dispersion serving as a condition for detecting behavior is set in a comparatively small range of values. On the other hand, in the case where it is assumed that the predetermined part that is included in the detection region is the shoulder region, the value of the dispersion serving as a condition for defecting behavior is set in a comparatively large range of values.

### (3) Non-Utilization of Foreground Region

In the above embodiment, the control unit 11 {information processing device 1) detects the behavior of the person being watched over utilising a foreground region that is extracted in step S202. However, the method of detecting the behavior of the person being watched over need not be limited to a method utilizing such a foreground region, and may be selected as appropriate according to the embodiment.

In the case of not utilizing a foreground region when detecting the behavior of the person being watched over, the control unit 11 may omit the processing of the above step S202. The control unit 11 may then function as the behavior detection unit 23, and detect behavior of the person being watched over that is related to the bed, by determining whether the positional relationship within real space between the bed reference plane and the person being watched over satisfies a predetermined condition, based on the depth for each pixel within the captured image 3. As an example of this, the control unit 11 may, as the processing of step S203, analyze the captured image 3 by pattern detection, graphic element detection or the like, and specify an image related to the person being watched over, for example. This image related to the person being watched over may be an image of the whole body of the person being watched over, and may be an image of one or more body parts such as the head and the shoulders. The control unit 11 may then detect behavior of the person being watched over that is related to the bed, based on the positional relationship within real space between the specified image related to the person being watched over and the bed.

Note that, as described above, the processing for extracting the foreground region is merely processing for calculating the difference between the captured image 3 and the background image. Thus, in the case of detecting the behavior of the person being watched over utilizing the foreground region as in the above embodiment, the control unit 11 (information processing device 1) will be able to detect the behavior of the person being watched over, without utilizing advanced image processing. It thereby becomes possible to accelerate processing relating to detecting the behavior of the person being watched over.

### (4) Non-Utilization of Depth Information

In the above embodiment, the control unit 11 (information processing device 1) detects the behavior of the person being watched over, by inferring the state of the person being watched over within real space based on depth information. However, the method of detecting the behavior of the person being watched over need not be limited to a method utilizing such depth information, and may be selected as appropriate according to the embodiment.

In the case of not utilizing depth information, the camera 2 need not include a depth sensor. In this case, the control unit 11 may function as the behavior detection unit 23, and detect the behavior of the person being watched over, by determining whether the positional relationship between the person being watched over and the bed that appear within the captured image 3 satisfies a predetermined condition. For example, the control unit 11 may analyze the captured image 3 by pattern detection, graphic element detection or the like to specify an image that is related to the person being watched over. The control unit 11 may then detect behavior of the person being watched over that is related to the bed, based on the positional relationship within the captured image 3 between the bed and the specified image that is related to the person being watched over. Also, for example, the control unit 11 may detect the behavior of the person being watched over, by determining whether the position at which the foreground region appears satisfies a predetermined condition, assuming that the target appearing in the foreground region is the person being watched over.

Note that, as described above, the position within real space of the subject appearing in the captured image 3 can be specified when depth information is utilized. Thus, in the case of detecting the behavior of the person being watched over utilizing depth information as in the above embodiment, the information processing device 1 becomes able to detect the behavior of the person being watched over with consideration for the state within real space.

### (5) Method of Setting Range of Bed Upper Surface

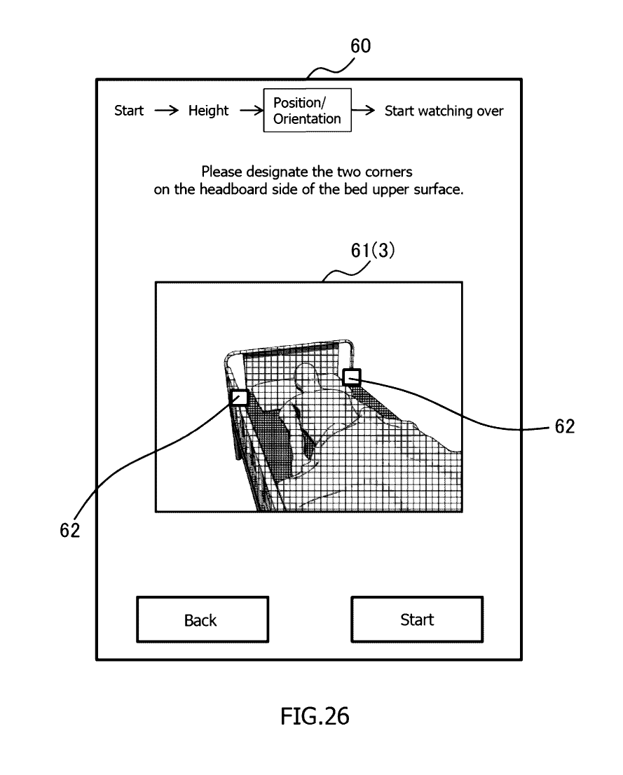

In step S105 of the above embodiment, the information processing device 1 (control unit 11) specified the range within real space of the bed upper surface, by accepting designation of the position of a reference point of the bed and the orientation of the bed. However, the method of specifying the range within real space of the bed upper surface need not be limited to such an example, and may be selected, as appropriate, according to the embodiment. For example, the information processing device 1 may specify the range within real space of the bed upper surface, by accepting specification of two corners out of the four corners defining the range of the bed upper surface. Hereinafter, this method will be described using FIG. 26.

FIG. 26 illustrates a screen 60 that is displayed on the touch panel display 13 when accepting setting of the range of the bed upper surface. The control unit 11 executes this processing in place of the processing of the above step S105. That is, the control unit 11 displays the screen 60 on the touch panel display 13, in order to accept designation of the range of the bed upper surface in step S105. The screen 60 includes a region 61 in which the captured image 3 obtained from the camera 2 is rendered, and two markers 62 for designating two corners out of the four corners defining the bed upper surface.

As described above, the size of the bed is often determined in advance according to the watching environment, and the control unit 11 is able to specify the size of the bed, using a set value determined in advance or a value input by a user. If the position within real space of two corners out of the four corners defining the range of the bed upper surface can be specified, the range within real space of the bed upper surface can be specified, by applying information {hereinafter, also referred to as the size information of the bed) indicating the size of the bed to the position of these two corners.

In view of this, the control unit 11 calculates the coordinates in the camera coordinate system of the two corners respectively designated by the two markers 62, with a method similar to the method used to calculate the coordinates P in the camera coordinate system of the reference point p designated by the marker 52 in the above embodiment, for example. The control unit 11 thereby becomes able to specify the position within real space of the two corners. On the screen 60 illustrated in FIG. 26, the user designates the two corners on the headboard side. Thus, the control unit 11 specifies the range within real space of the bed upper surface by treating these two corners specifying positions within real space as the two corners on the headboard side, and estimating the range of the bed upper surface.

For example, the control unit 11 specifies the orientation of a vector connecting these two corners whose position was specified within real space as the orientation of the headboard. In this case, the control unit 11 may treat one of the corners as the starting point of the vector. The control unit 11 then specifies the orientation of a vector facing toward the perpendicular direction at the same height as the above vector as the direction of the side frame. In the case where there are a plurality of candidates as the direction of the side frame, the control unit 11 may specify the direction of the side frame in accordance with a setting determined in advance, or may specify the direction of the side frame based on a selection by the user.

Also, the control unit 11 associates the length of the lateral width of the bed that is specified from the size information of the bed with the distance between the two corners whose position was specified within real space. The scale in the coordinate system (e.g., camera coordinate system) representing real space is thereby associated with real space. The control unit 11 then specifies the position within real space of the two corners on the footboard side that exist, in the direction of the side frame from the respective two corners on the headboard side, based on the length of the longitudinal width of the bed specified from the size information of the bed. The control unit 11 is thereby able to specify the range within real space of the bed upper surface. The control unit 11 sets the range that, is thus specified as the range of the bed upper surface. Specifically, the control unit 11 sets the range that, is specified based on the position of the markers 62 that had been designated when a “start” button was operated as the range of the bed upper surface.

Note that, in FIG. 26, the two corners on the headboard side are illustrated as the two corners for accepting designation. However, the two corners for accepting designation need not be limited to such an example, and may be suitably selected from the four corners defining the range of the bed upper surface.

Also, designation of the positions of which of the four corners defining the range of the bed upper surface to accept may be determined in advance as described above or may be decided by a user selection. This selection of the corners whose position is to be designated by the user may be performed before specifying the position or may be performed after specifying the positions.

Furthermore, the control unit 11 may render, within the captured image 3, the frame FD of the bed that, is specified from the position of the two markers that have been designated, similarly to the above embodiment. By thus rendering the frame FD of the bed within the captured image 3, it is possible to allow the user to check the range of the bed that has been designated, together with allowing the user visually confirm by sight which corners to designate.

### (6) Other Matters

Note that the information processing device I according to the embodiment calculates various values relating to setting of the position of the bed, based on relational equations that take the pitch angle a of the camera 2 into consideration. However, the attribute value of the camera 2 that the information processing device 1 fakes into consideration need not be limited to this pitch angle a, and may be selected, as appropriate, according to the embodiment. For example, the information processing device 1 may calculate various values relating to setting of the position of the bed, based on relational equations that take the roll angle of the camera 2 and the like into consideration in addition to the pitch angle α of the camera 2.

Also, the reference plane of the bed that serves as a reference for the behavior of the person being watched over may be set in advance, independently of the above steps S103 to step S108. The reference plane of the bed may be set, as appropriate, according to the embodiment. Furthermore, the information processing device 1 according to the embodiment may determine the positional relationship between the target appearing in the foreground region and the bed, independently of the reference plane of the bed. The method of determining the positional relationship between the target appearing in the foreground region and the bed may be set, as appropriate, according to the embodiment.

Also, in the above embodiment, the instruction content for aligning the orientation of the camera 2 with the bed is displayed within the screen 40 for setting the height of the bed upper surface. However, the method of displaying the instruction content for aligning the orientation of the camera 2 with the bed need not be limited to such a mode. The control unit 11 may cause the touch panel display 13 to display the instruction content for aligning the orientation of the camera 2 with the bed and the captured image 3 that is acquired by the camera 2 on a separate screen to the screen 40 for setting the height of the bed upper surface. Also, the control unit 11 may accept, on that screen, that adjustment of the orientation of the camera 2 has been completed. The control unit 11 may then cause the touch panel display 13 to display the screen 40 for setting the height of the bed upper surface, after accepting adjustment of the orientation of the camera 2 has been completed.

### REFERENCE SIGNS LIST

1 Information processing device

2 Camera

3 Captured image

5 Program

6 Storage medium

21 Image acquisition unit

22 Foreground extraction unit

23 Behavior detection unit

24 Setting unit

25 Display control unit

2 6 Behavior selection unit

27 Danger indication notification unit

28 Non-completion notification unit

1. An information processing device comprising:
a behavior selection unit configured to accept selection of behavior to be watched for with regard to a person being watched over, from a plurality of types of behavior, related to a bed, of the person being watched over;
a display control unit configured to cause a display device to display a candidate arrangement position, with respect to the bed, of an image capturing device for watching for behavior, in the bed, of the person being watched over, according to the behavior selected to be watched for;
an image acquisition unit configured to acquire a captured image captured by the image capturing device; and
a behavior detection unit configured to detect the behavior selected to be watched for, by determining whether a positional relationship between the bed and the person being watched over appearing in the captured image satisfies a predetermined condition.

2. The information processing device according to claim 1,
wherein the display control unit causes the display device to further display a preset position where installation of the image capturing device is not recommended, in addition to the candidate arrangement position of the image capturing device with respect to the bed.

3. The information processing device according to claim 1,
wherein the display control unit, after accepting that arrangement of the image capturing device has been completed, causes the display device to display the captured image acquired by the image capturing device, together with instruction content for aligning orientation of the image capturing device with the bed.

4. The information processing device according to claim 1,
wherein the image acquisition unit acquires a captured image including depth information indicating a depth for each pixel within the captured image, and
the behavior detection unit detects the behavior selected to be watched for, by determining whether a positional relationship within real space between the person being watched over and a region of the bed satisfies a predetermined condition, based on the depth for each pixel within the captured image that is indicated by the depth information, as the determination of whether the positional relationship between the bed and the person being watched over appearing in the captured image satisfies a predetermined condition.

5. The information processing device according to claim 4, further comprising:
a setting unit configured to, after accepting that arrangement of the image capturing device has been completed, accept designation of a height of a reference plane of the bed, and sets the designated height as the height of the reference plane of the bed,
wherein the display control unit, when the setting unit is accepting designation of the height of the reference plane of the bed, causes the display device to display the captured image that is acquired, so as to clearly indicate, on the captured image, a region capturing a target located at the height designated as the height of the reference plane of the bed, based on the depth for each pixel within the captured image that is indicated by the depth information, and
the behavior detection unit detects the behavior selected to be watched for, by determining whether a positional relationship between the reference plane of the bed and the person being watched over in a height direction of the bed within real space satisfies a predetermined condition.

6. The information processing device according to claim 5, further comprising:
a foreground extraction unit configured to extract a foreground region of the captured image from a difference between the captured image and a background image set as a background of the captured image.
wherein the behavior detection unit detects the behavior selected to be watched for, by determining whether the positional relationship between the reference plane of the bed and the person being watched over in the height direction of the bed within real space satisfies a predetermined condition, utilizing, as a position of the person being watched over, a position within real space of a target appearing in the foreground region that is specified based on the depth for each pixel within the foreground region.

7. The information processing device according to claim 5.
wherein the behavior selection unit accepts selection of behavior to be watched for with regard to the person being watched over, from a plurality of types of behavior, related to the bed, of the person being watched over that include predetermined behavior of the person being watched over that is carried out in proximity to or on an outer side of an edge portion of the bed,
the setting unit accepts designation of a height of a bed upper surface as the height of the reference plane of the bed and sets the designated height as the height of the bed upper surface, and, in a case where the predetermined behavior is included in the behavior selected to be watched for, further accepts, after setting the height of the bed upper surface, designation, within the captured image, of an orientation of the bed and a position of a reference point that is set within the bed upper surface in order to specify a range of the bed upper surface, and sets a range within real space of the bed upper surface based on the designated orientation of the bed and position of the reference point, and
the behavior detection unit detects the predetermined behavior selected to be watched for, by determining whether a positional relationship within real space between the set upper surface of the bed and the person being watched over satisfies a predetermined condition.

8. The information processing device according to claim 5,
wherein the behavior selection unit accepts selection of behavior to be watched for with regard to the person being watched over, from a plurality of types of behavior, related to the bed, of the person being watched over that include predetermined behavior of the person being watched over that is carried out in proximity to or on an outer side of an edge portion of the bed,
the setting unit accepts designation of a height of a bed upper surface as the height of the reference plane of the bed and setting the designated height as the height of the bed upper surface, and, in a case where the predetermined behavior is included in the behavior selected to be watched for, further accepts, after setting the height of the bed upper surface, designation, within the captured image, of positions of two corners out of four corners defining a range of the bed upper surface, and sets a range within real space of the bed upper surface based on the designated positions of the two comers, and
the behavior detection unit detects the predetermined behavior selected to be watched for, by determining whether a positional relationship within real space between the set upper surface of the bed and the person being watched over satisfies a predetermined condition.

9. The information processing device according to claim 7,
wherein the setting unit determines, with respect to the set range of the bed upper surface, whether a detection region specified based on the predetermined condition set in order to detect the predetermined behavior selected to be watched for appears within the captured image, and, in a case where it is determined that the detection region of the predetermined behavior selected to be watched for does not appear within the captured image, outputs a warning message indicating that there is a possibility that detection of the predetermined behavior selected to be watched for cannot be performed normally.

10. The information processing device according to claim 7, further comprising:
a foreground extraction unit configured to extract a foreground region of the captured image from a difference between the captured image and a background image set as a background of the captured image,
wherein the behavior defection unit detects the predetermined behavior selected to be watched for, by determining whether a positional relationship within real space between the bed upper surface and the person being watched over satisfies a predetermined condition, utilizing, as a position of the person being watched over, a position within real space of a target appearing in the foreground region that is specified based on the depth for each pixel within the foreground region.

11. The information processing device according to claim 5, further comprising:
a non-completion notification unit configured to, in a case where setting by the setting unit is not completed within a predetermined period of time, perform notification for informing that setting by the setting unit has not been completed.

12. An information processing method in which a computer executes:
a step of accepting selection of behavior to be watched for with regard to a person being watched over, from a plurality of types of behavior, related to a bed of the person being watched over;
a step of causing a display device to display a candidate arrangement position, with respect to the bed, of an image capturing device for watching for behavior, in the bed, of the person being watched over, according to the behavior selected to be watched for;
a step of acquiring a captured image captured by the image capturing device; and
a step of detecting the behavior selected to be watched for, by determining whether a positional relationship between the bed and the person being watched over appearing in the captured image satisfies a predetermined condition.

13. A non-transitory recording medium recording a program to cause a computer to execute;
a step of accepting selection of behavior to be watched for with regard to a person being watched over, from a plurality of types of behavior, related to a bed, of the person being watched over;
a step of causing a display device to display a candidate arrangement position, with respect to the bed, of an image capturing device for watching for behavior, in the bed, of the person being watched over, according to the behavior selected to be watched for;
a step of acquiring a captured image captured by the image capturing device; and
a step of detecting the behavior selected to be watched for, by determining whether a positional relationship between the bed and the person being watched over appearing in the captured image satisfies a predetermined condition.
